(19) United States
(12) Patent Application Publication
(10) Pub. No.: US 2013/0177567 A1
(43) Pub. Date: Jul. 11, 2013

(54) NOVEL C3C EPITOPE, ANTIBODIES BINDING THERETO, AND USE THEREOF

(71) Applicants: Mikkel Ole Skjoedt, Frederiksberg C (DK); Lars Vitved, Odense C (DK); Yaseelan Palarasah, Odense C (DK)

(72) Inventors: Mikkel Ole Skjoedt, Frederiksberg C (DK); Lars Vitved, Odense C (DK); Yaseelan Palarasah, Odense C (DK)

(73) Assignee: Sydansk Universitet, Odense M (DK)

(21) Appl. No.: 13/698,614

(22) PCT Filed: May 17, 2011
(86) PCT No.: PCT/EP2011/058007

§ 371 Date: Feb. 20, 2013

Related U.S. Application Data
(60) Provisional application No. 61/345,816, filed on May 18, 2010.

(51) Int. Cl. C07K 16/18; C07K 7/08; C07K 7/06; G01N 33/68

(52) U.S. Cl. CPC C07K 16/18; G01N 33/6893; C07K 7/08; C07K 7/06
USPC 424/139.1; 530/387.9; 435/7.94; 435/331; 530/326; 530/328

(57) ABSTRACT

Provided are antibodies directed against a novel epitope on the human C3c (complement factor 3 c) and the use and manufacture thereof. Moreover, there is provided a novel immunogen for use in the preparation of the antibodies. The antibodies are in the first place of the monoclonal type.

### FIELD OF THE INVENTION

The present invention relates to antibodies directed against a novel epitope on the human C3c (the c portion of the third complement factor) and the use and manufacture thereof. Moreover, the present invention relates to a novel immunogen for use in the preparation of the antibodies of the present invention. The antibodies of the present invention are in the first place of the monoclonal type.

### BACKGROUND OF THE INVENTION

The complement system involves a large number of plasma proteins that react with one another in a sequential order to opsonize or directly kill invading micro-organisms, and to contribute to the induction of inflammatory responses [1, 2]. A broad range of diseases is characterized by the involvement of a systemic inflammatory response. Such diseases are among others autoimmune diseases like systemic lupus erythematosus (SLE), rheumatoid arthritis and multiple sclerosis [3, 4]. Also reactions due to transplantation, certain cardiovascular diseases, and infectious diseases may involve a systemic inflammatory response [5]. In the light of this, components of the complement system are obvious biomarkers for systemic inflammation in acute and chronic diseases.

The central component of the complement system is C3 (186 kDa) consisting of a β-chain (75 kDa) and an α-chain (110 kDa) connected by cystein bridges. Cleavage of C3 into C3b (177 kDa) and C3a (9 kDa) is a pivotal step in the complement activation cascade, which can be initiated by three distinct pathways—the classical, the lectin and the alternative complement pathway. As a result of activation C3b covalently attaches to foreign surfaces (e.g a microorganism) to immunecomplexes or to apoptotic target surfaces via its reactive thioester moiety and thereby induces several biological processes such as further activation of C3 and activation of C5, which will subsequently result in assembly of the membrane attack complex, C5b,6,7,8,9 [6]. Finally, C3b undergoes successive proteolytic cleavages leading to inactive C3-products. These steps are mediated by the regulatory enzyme factor I and lead to generation of iC3b (174 kDa), C3d (equal to C3dg, 33 kDa) C3f (2 kDa) and C3c (142 kDa).

A measurement of such cleavage products will thus indicate the degree of complement activation and provide a better basis for prognosis and decisions about treatment. Various complement assays have been developed and are used in the clinic. These include the measurement of the functional level of complement capacity like in haemolytic assays (CHSO), or in recently developed functional ELISA-based assays [7, 8]. Other assays measures fluid phase terminal complex (C5b-C9) by ELISA-techniques, and precipitation-in-gel techniques has been used to quantify split products (e.g. C3d) [9, 10]. Most of these methods are elaborate, time consuming and difficult to standardize and for the capacity based assays rely on serum source with ex vivo complement activation. Other approaches have been assessment of the anaphylatoxins C3a and C5a, but these fragments have extremely short half-lives and can be undetectable [11, 12].

Skjødt et al [22] disclose a panel of mouse monoclonal antibodies (mAbs) that are able to detect fluid phase C3c without interference from other products generated from the third complement component factor C3. These antibodies form the basis for the generation of assays for quick, reliable and cost efficient evaluation of complement activation and consumption as a marker for inflammatory processes. The paper states that C3c is a reliable acute phase marker and in comparison with C3d since it has a short half-life time and does not bind to other components (cell surfaces, serum proteins etc.), which might interfere with the assay. Meanwhile Skjødt et al [22] do not disclose an assay for the selective detection of the subcomponent C3c of the complement system and thus for inflammatory processes.

Hugo et al [23] disclose the production and characterization of mouse monoclonal antibodies to C9-dependent neoantigens of human C5b-9. Binding-inhibition assays with EDTA-human plasma and micro-ELISA assays with purified C9 are also disclosed showing that the antibodies does not react with native complement components and thus confirmed the specificity of the antibodies for the neoantigens. The monoclonal antibodies did, however, cross-react with cytolytically inactive, fluid-phase C5b-9 complexes. Hugo et al [23] assess the serum C5b-C9 levels in women with endometriosis compared with controls. In the women with endometriosis, higher levels of C5b-C9 in serum were observed for the advanced stages of disease in comparison with early stages. Hugo et al [23] do not address the specific/selective C3c monoclonal antibodies.

Henwick et al [24] disclose monoclonal antibodies, which recognize specific C3 fragments useful to distinguish C3 cleavage products bound to organisms. The authors defined the specificity of three commercially available monoclonal antibodies by Western immunoblot analysis, enzyme-linked immunosorbent assay, and a quantitative flow cytometric technique. Two monoclonal antibodies with specificity for (i) an erythrocyte-bound C3d epitope or (ii) an erythrocyte-bound C3c epitope retained their specificity in all assays. However, C3c is not bound to erythrocytes covalently or via complement receptors due to the absence of the thioester moiety and the domains responsible for the binding to erythrocyte receptors. Presence of C3 derivatives on erythrocytes is expected to be in the forms of C3b, iC3b and C3d. The antibodies disclosed in this study will thus presumably react with the C portion of C3b, iC3b and C3c.

U.S. Pat. No. 4,960,712 discloses systems and methods used to assay for particular complement component fragments. This can be used to determine the amount of a particular complement component fragment in a sample. The fragment can be fluid phase or bound to an immune complex. Generally, specific binding agents, such as antibodies, directed to the complement component fragments and immune complexes are used in the assay. For instance a second binding agent specific for C3c is also reacted with at least a portion of the sample and the amount of binding by this agent is also measured. Meanwhile the second binding agent specific to C3c reacts with C3b and iC3b. The antibodies discussed in this patent do not exclusive bind C3c (or fragments thereof).

There is a general need for markers for systemic inflammation in acute or chronic diseases, and factors of the complement system are obvious targets. Available methods today are of low sensitivity and are both elaborate and time consuming; they include haemolytic assays (CH50), quantification of terminal complex (C5b-C9) and quantification of split products in precipitation-in-gel techniques (e.g. C3d).

It has been speculated that the C3c fragment consisting of the β-chain, the α₂ (40 kDa) and α₃ (27 kDa) domain, would be a good candidate, since C3c unlike other C3 derivates does not bind to other structures (foreign pathogens, cell surface receptors, other plasma proteins, etc.). Hence, it would be relevant to use in vivo generated plasma C3c as an indicator of the inflammatory state.

Accordingly, it is an object of the present invention to provide a monoclonal antibody (mAb), which is able to detect fluid phase C3c without interference from intact C3 or other products generated from C3. The C3c specific mAb should not cross-react with intact uncleaved C3, C3b, iC3b or with C3d.

### SUMMARY OF THE INVENTION

The present invention is directed to antibodies (Abs), especially monoclonal antibodies (mAbs), which are able to detect fluid phase C3c without interference from intact C3 or other products generated from C3. The present invention is also directed to the specific epitope of C3c to which the antibodies of the present invention bind to. Furthermore the present invention provides an immunogen against which the antibodies of the present invention are raised.

The C3c specific mAbs have been tested in a variety of combinations in order to demonstrate that it does not cross-react with intact uncleaved C3, C3b, iC3b or with C3d. By using these antibodies as the capture reagent in a sandwich ELISA an assay has been established, whereby the distribution range of C3c among a panel of human blood donors can be determined.

The present inventors have surprisingly found that the novel epitope of C3c having the amino acid sequence CLDPERLGR (SEQ ID NO 1) is particularly useful for the selective detection of C3c and important fragments. The present inventors have also prepared a novel immunogen with the amino acid sequence NKTVAVRTLDPERLGR (SEQ ID NO 2), which is useful for raising antibodies that specifically bind to the epitope (SEQ ID NO 1) of the present invention. Hence, protection is herewith sought for the isolated sequences CLDPERLGR (SEQ ID NO 1) and NKTVAVRTLDPERLGR (SEQ ID NO 2). Additionally the antibodies of this present invention do not bind to the immunogens CLDPERLGR (SEQ ID NO 1) and NKTVAVRTLDPERLGR (SEQ ID NO 2) if the native carboxy-terminal end is chemically modified to an amidated end residue.

The preferred antibody preparations of the present invention are not significantly inhibited in their reaction with C3c by soluble (free) native C3b, C3bi, C3d,g or CM fragments. For instance under the conditions given in the experimental part a more than 25 times higher, such as 50 times higher dose of the soluble fragments compared to the corresponding SDS-denatured or covalently bound fragments (molar basis) is required to effect the identical inhibition in an inhibition ELISA.

Specifically, in one aspect the present invention concerns an antibody directed against an individual epitope in the C3c region of C3, characterized by immunochemically reacting with an epitope in the C3c region of native C3, said epitope having the sequence of SEQ ID NO 1.

In another aspect the present invention is directed to a monoclonal antibody specific to the epitope of the C3c region of human C3 having the sequence of SEQ ID NO 1.

In still another aspect the present invention is directed to the use of the monoclonal antibody of the present invention for measurement of C3c concentration in a biological sample.

In a further aspect the present invention is directed to the use of the monoclonal antibody of the present invention for measurement in body fluids, such as in serum. This use may be achieved with a multi analytical assay.

The present invention also concerns a method for immunoassay detection and quantification of C3c comprising the steps of: 1) contacting a sample containing C3c with an antibody of the present invention to form a complex between the antibody and C3c in an amount that is related to the amount of C3c, and 2) determining the amount of complex formed and relating the amount found to the amount of C3c in the sample.

The present invention also provides a therapeutic composition comprising a therapeutically effective amount of the antibody of the present invention in a pharmaceutically acceptable carrier substance.

The present invention is also directed to a method for treating of a patient with a need to decrease the amount of C3c by administering the antibody of the present invention.

In a further aspect the present invention provides a hybridoma cell line which produces a monoclonal antibody of the present invention.

Importantly the present invention provides a composition of matter consisting essentially of polyclonal antibodies, which specifically bind to C3c, and which have less than 20% cross reactivity with C3, C3b or iC3b. wherein said polyclonal antibodies are produced by immunizing an animal with the immunogen having the sequence of SEQ ID NO 2.

The present invention also contemplates the use of an antibody of the present invention for immunochemically binding C3c fragments bearing the epitope against which the antibody is directed.

Moreover, the present invention is directed to the production of an antibody possessing specificity for the epitope in the C3c region human native C3 having the sequence of SEQ ID NO 1, characterized in that cells potentially capable of producing antibodies are caused to secrete the antibody whereupon the antibody is purified and optionally isolated and/or derivatized.

The antibody of the present invention has potential value as a reagent in the assessment of in vivo complement activity during inflammatory processes. In a broader perspective the present inventors have found that the present invention can be used in the assessment of the following patient groups:

Patients in an acute phase:
Samples from patients with suspected systemic infection. Including patients with high CRP levels. Included sepsis patients and patient with systemic inflammatory response syndrome (SIRS).
Transplanted patients (with and without GvH/HvG)
Sepsis
Anaphylatoxic shock
Ischemia reperfusion injuries
Kidney failure
Liver failure
Follow up during surgery
Pre-eclampsia

### Patients in a Chronic Phase:

### Autoimmune Diseases Such As:

Systemic lupus erythematosus (SLE), rheumatoid arthritis, juvenile arthritis, Type 1 diabetes, Morbus Bechterew etc
Allergies
Crohns disease (Morbus Crohn)

### Functional Hereditary Complement Deficiencies Such as:

Factor I, factor H, Properdin, Mannose binding lectin deficiencies And C1 inhibitor deficiency resulting in hereditary angioedema (HAE)

### CNC Diseases Such As:

Parkinsons, amyotrophic lateral sclerosis, Huntington's, Tourettes, multiple sclerosis etc

### BRIEF DESCRIPTION OF THE DRAWINGS

FIG. 1 shows immunoprecipitation of C3 products.

FIG. 2 shows purification of C3 derivates.

FIG. 3 shows deposited C3 on a solid phase immune-complex surface (ICS).

FIG. 4 shows C3c levels in serum and plasma samples measured by a C3c specific sandwich ELISA.

FIG. 5 shows calibration curves and parallelism.

FIG. 6 shows the C3c range in plasma from 100 Danish blood donors and the C3c level in plasma samples from two different factor I-deficient patients.

FIG. 7 A shows stability and recovery tests.

FIG. 7 B shows effect of storage.

FIG. 7 C shows analysis of the intra-assay and inter-assay variation.

FIG. 8 shows assessment of the C3c level in four factor H deficient patients.

FIG. 9 shows assessment of the C3c level in 18 patients with elevated CRP and suspicion of a systemic infection.

FIG. 10 shows assessment of the C3c level in 18 patients with elevated CRP and suspicion of a systemic infection.

FIG. 11 shows C3c plasma levels in patients with chronic obstructive lung disease (COLD).

FIG. 12 shows C3c plasma levels in patients with functional factor H deficiency.

FIGS. 13A-G show plasma C3c levels in patients undergoing endovascular surgery.

### DETAILED DESCRIPTION OF THE INVENTION

The antibodies of the present invention may be present in the form of antibody-active fragments, for instance Fab, Fab′, and F(ab) 2*. They may also be in the form of derivatized antibodies. The essential requirement is that the antibody fragments and the derivatives possess biospecific immunetype affinity in accordance with the present invention.

The antibody-active components of the preparations may be provided with analytically detectable groups such as enzymatically active, fluorogenic, chemiluminogenic, radioactive, biotinyl groups etc., or groups capable of acting as cofactors, coenzymes, substrates or co-substrates etc.

For producing an antibody preparation according to the invention, cells potentially capable of producing antibodies of the specificity as prescribed in conformity with the invention are caused to secrete such antibodies which are then isolated, purified and optionally fragmentized and/or derivatized. The purification involves removal of those antibodies that fail to fulfill the prescribed specifications. In a vertebrate, preferably a warm-blooded vertebrate (e. g. mammal such as mouse) secretion may take place in vivo as a result of immunization with an immunogen having the particular neoantigenic structure (epitope) as contemplated here. The resultant immune response is polyclonal, so an antiserum obtained from the animal will contain antibodies directed against all the determinants of the immunogen, irrespective of whether or not these are neoantigenic. By employing suitable selection methods the specificity of the immune response can be limited. By means of a suitable immunosorbent technique it is potentially possible to obtain purified forms of the antibodies directed against the desired neoantigen. In the present stage immunosorbent purification of polyclonal antibodies to obtain the preparation of the invention is a laborious procedure which will always give low yields.

The best method for selecting the antibodies in the immune response in order to obtain a good antibody preparation according to the invention is a monoclonal technique by which after immunization antibody-producing plasma cells are fused with cells of a suitable myeloma cell line so that they become capable of quick and uninterrupted growth. By cloning, selecting and culturing the fused cells that produce antibodies having the specificity (C3c) and low cross reactivity in accordance with this invention it is possible to obtain antibody preparations directed against the epitope of the present invention. Cultivation of the selected cell clones for producing the antibody preparations of the invention may be carried out in cell cultures in vitro or as ascites tumors in vivo. Purification and isolation may be performed in the same manner as purification and isolation of any antibodies in general—by salt precipitation or by means of various chromatographic methods like, for instance, ion exchange, affinity, or gel chromatography.

The antibody preparations of the present invention are useful primarily in immunochemical assay methodology for C3c fragments, but they may also be used potentially both in vitro and in vivo for modulating complement activation.

The assays contemplated in the present context involve contacting a sample containing C3c with the antibody preparations of this invention to thus form an immune complex the formation and amount of which are a quantitative measure and qualitative measure, respectively, of the C3c in the sample.

The invention is applicable to assays in various types of samples which contain C3c and/or fragments thereof. It has been shown that C3c and its fragments are present in, for example, tissues and body fluids like blood, plasma, serum, urine, synovial fluid, cerebrospinal fluid etc.

The invention is further defined in the attached claims and will now be illustrated by means of examples which form a part of this specification and which are not to be construed as limiting the scope of the invention in any way.

### EXAMPLE 1

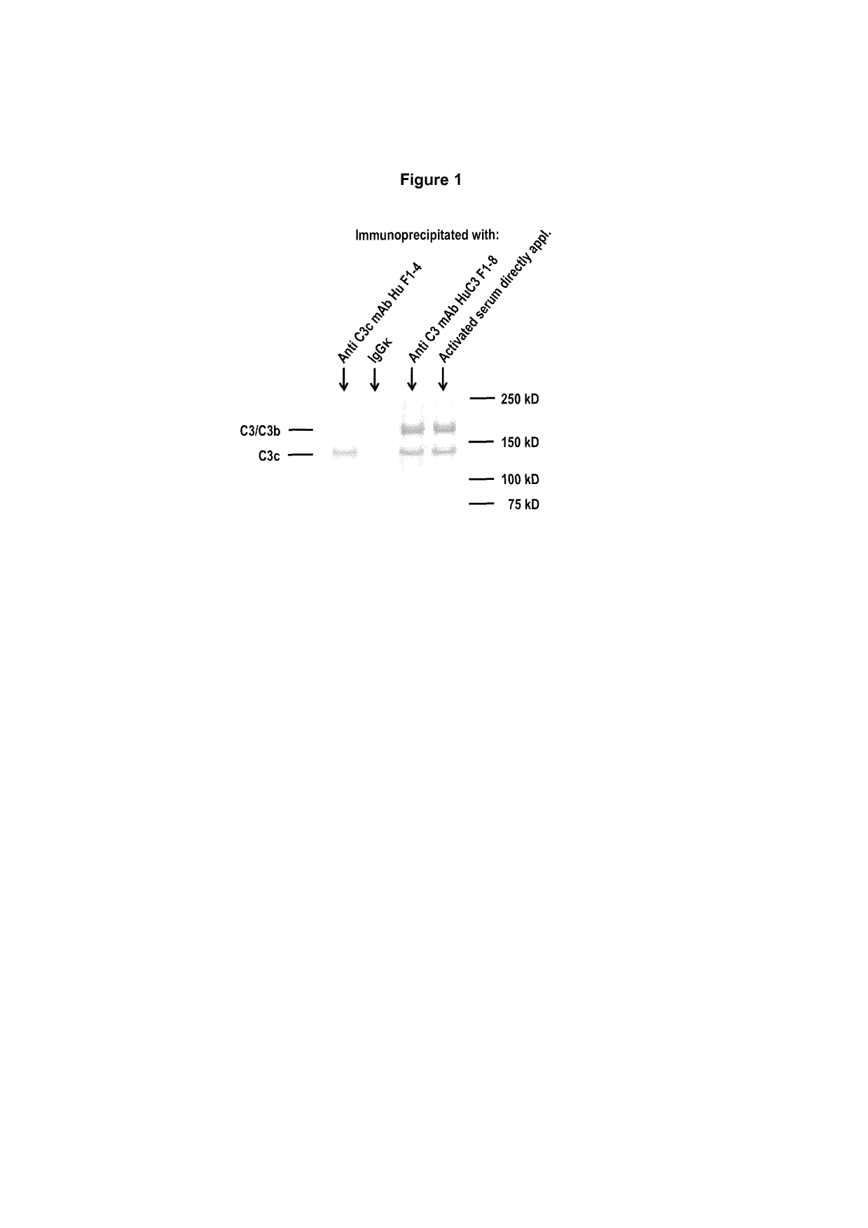

In FIG. 1 immunoprecipitation of C3 products is shown. The C3c specific mAb HuC3c F1-4 and anti C3 mAb HuC3 F1-8 were used to immunoprecipitate C3 derivates from activated serum. Non-reduced samples were electro-blotted and developed with polyclonal antibodies to C3c and C3d. The molecular marker was Precision Plus Protein™ Prestained (B10 RAD).

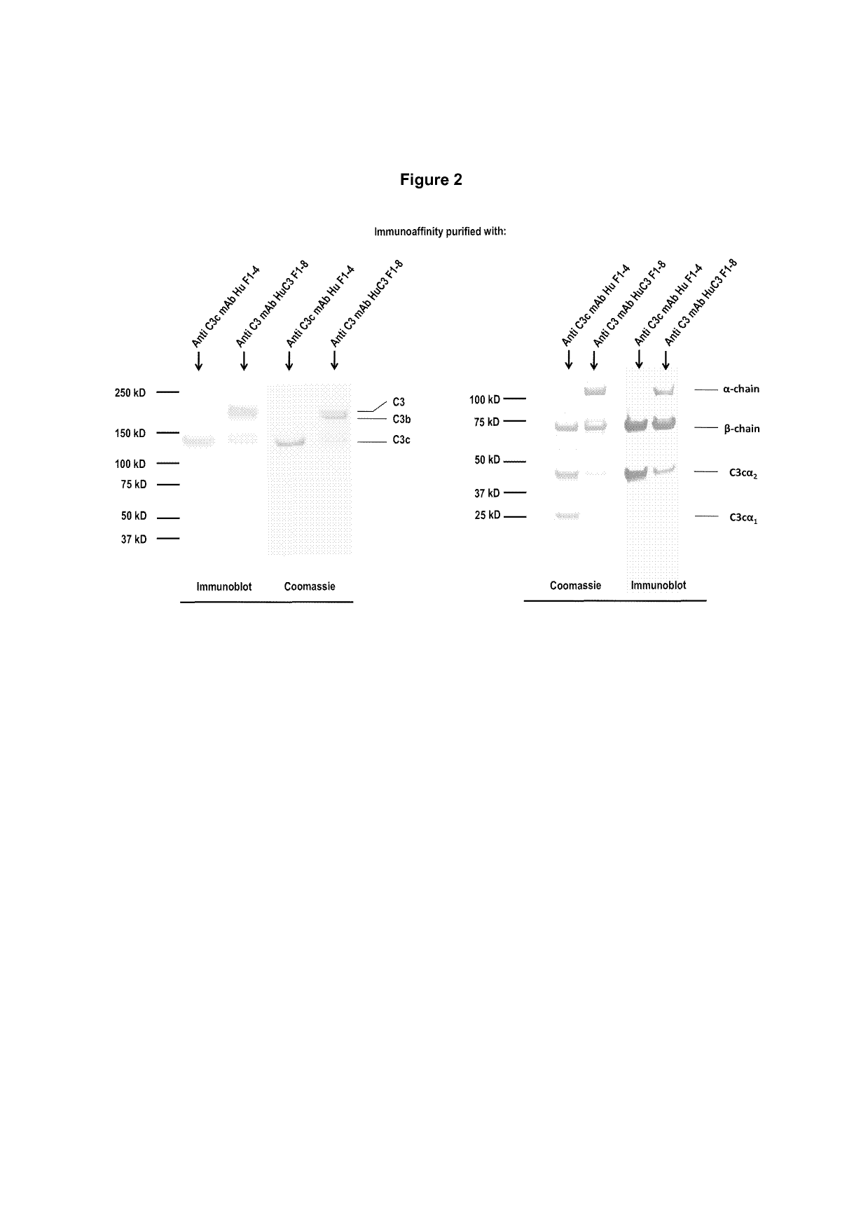

In FIG. 2 urification of C3 derivates is shown. C3 derivatives from in vitro activated NHS was purified with C3c specific mAb HuC3c F1-4 or anti mAb HuC3 F1-8 columns. The eluted fractions were analyzed by reducing and non-reducing SDS-PAGE and coomassie staining or immunoblotting with a mixture of polyclonal anti C3c and C3d as detection antibodies. Non-reduced fractions analyzed by coomassie staining and immunoblotting (left side). Reduced fractions analyzed by coomassie staining and immunoblotting (right side).

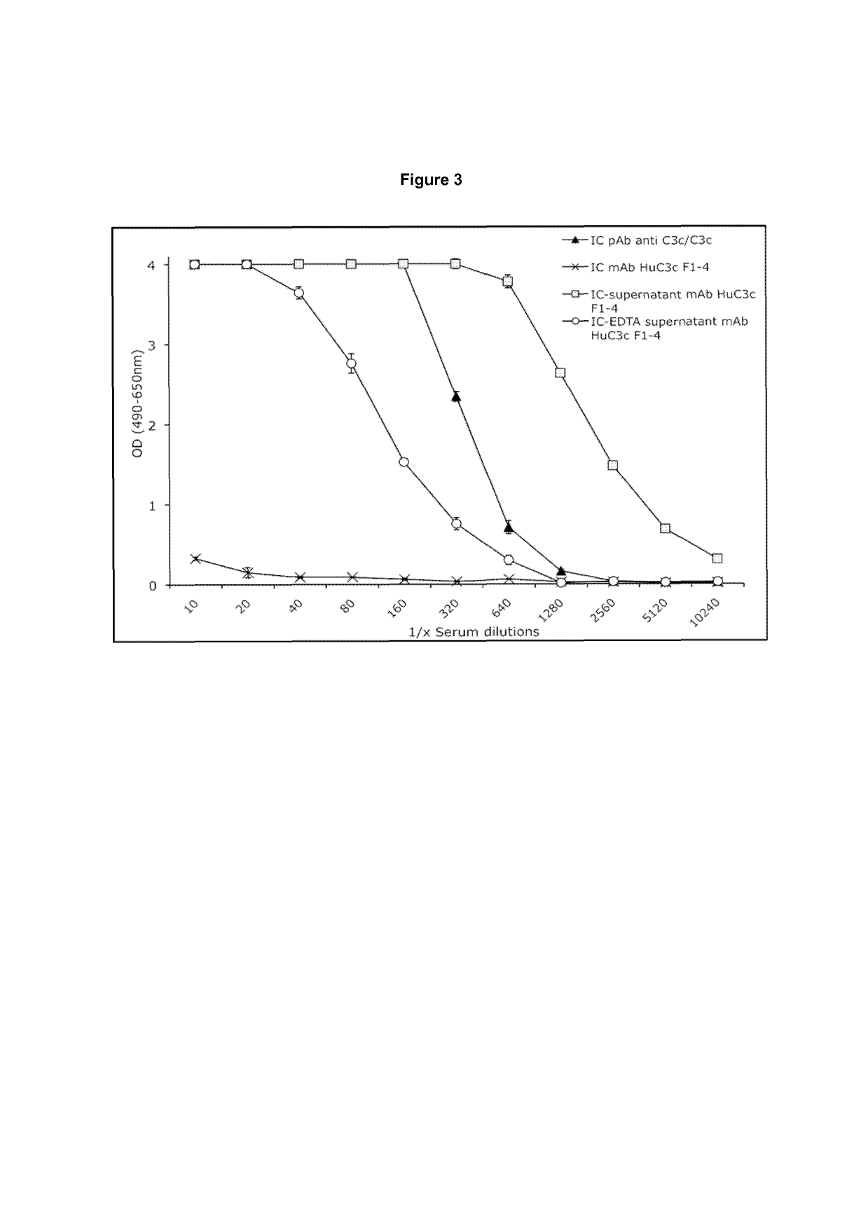

In FIG. 3 deposited C3 on a solid phase immune-complex surface (IC) is shown. Two-fold serum dilutions were incubated on an IC. Activation and deposition of iC3b, C3b and C3d on the IC were assessed using a mixture of biotinylated polyclonal anti-human C3c and C3d (IC pAb anti C3c/d IC) or C3c specific mAb HuC3c F1-4 (IC mAb HuC3c F1-4). Supernatant after the IC incubation was assessed in a C3c sandwich ELISA using mAb HuC3c F1-4 as catching antibody (IC supernatant mAb HuC3c F1-4). EDTA plasma incubated on the IC served as a control for base line C3c level (IC EDTA supernatant mAb HuC3c F1-4). The levels are given as OD_{490-650 nm}-units. Error bars indicate two times the standard deviation of double determinations.

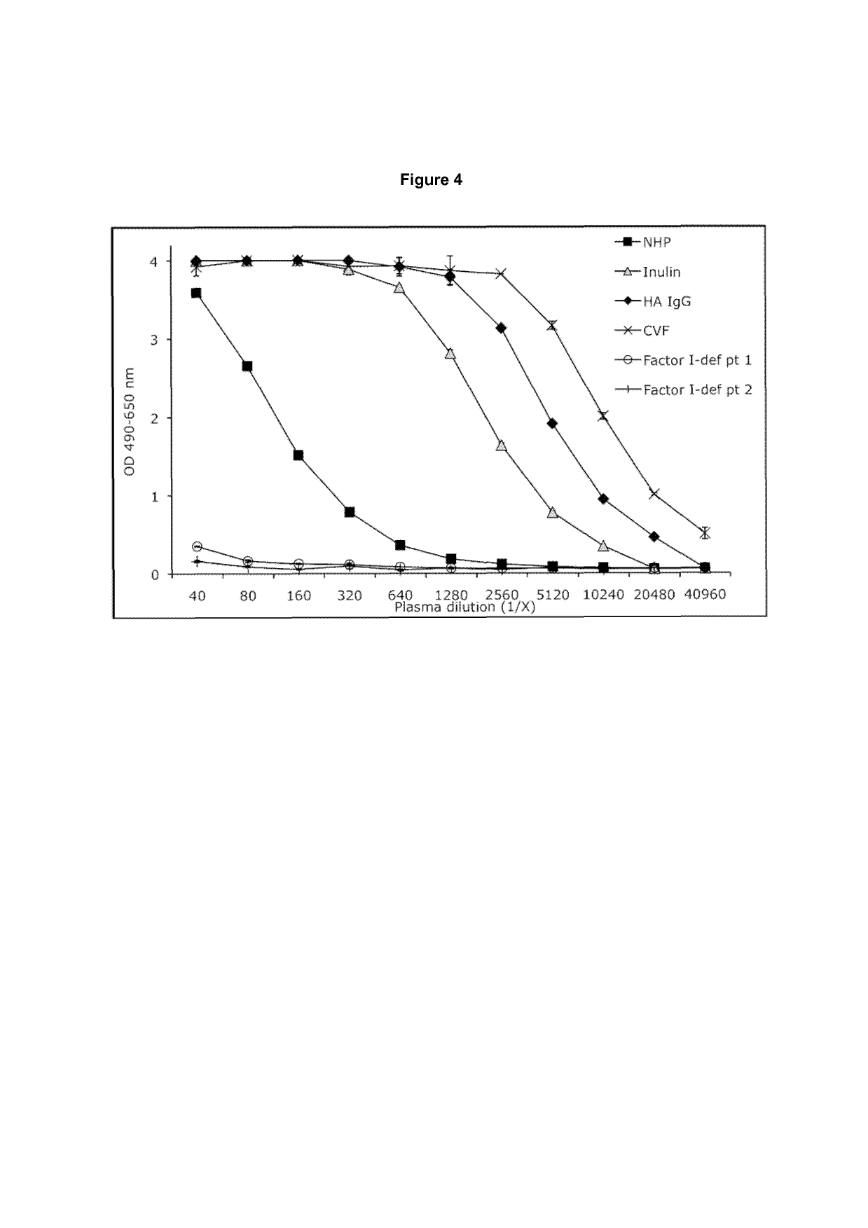

In FIG. 4 C3c levels in serum and plasma samples measured by a C3c specific sandwich ELISA is shown. Serum was in vitro activated with inulin, heat aggregated IgG (HA IgG) or Cobra venom factor (CVF). After activation sera were made 10 mM with respect to EDTA. The levels are given as OD_{490-650 nm}-units. The controls were none activated plasma and factor I deficient plasma. Error bars indicate two times the standard deviation of double determinations.

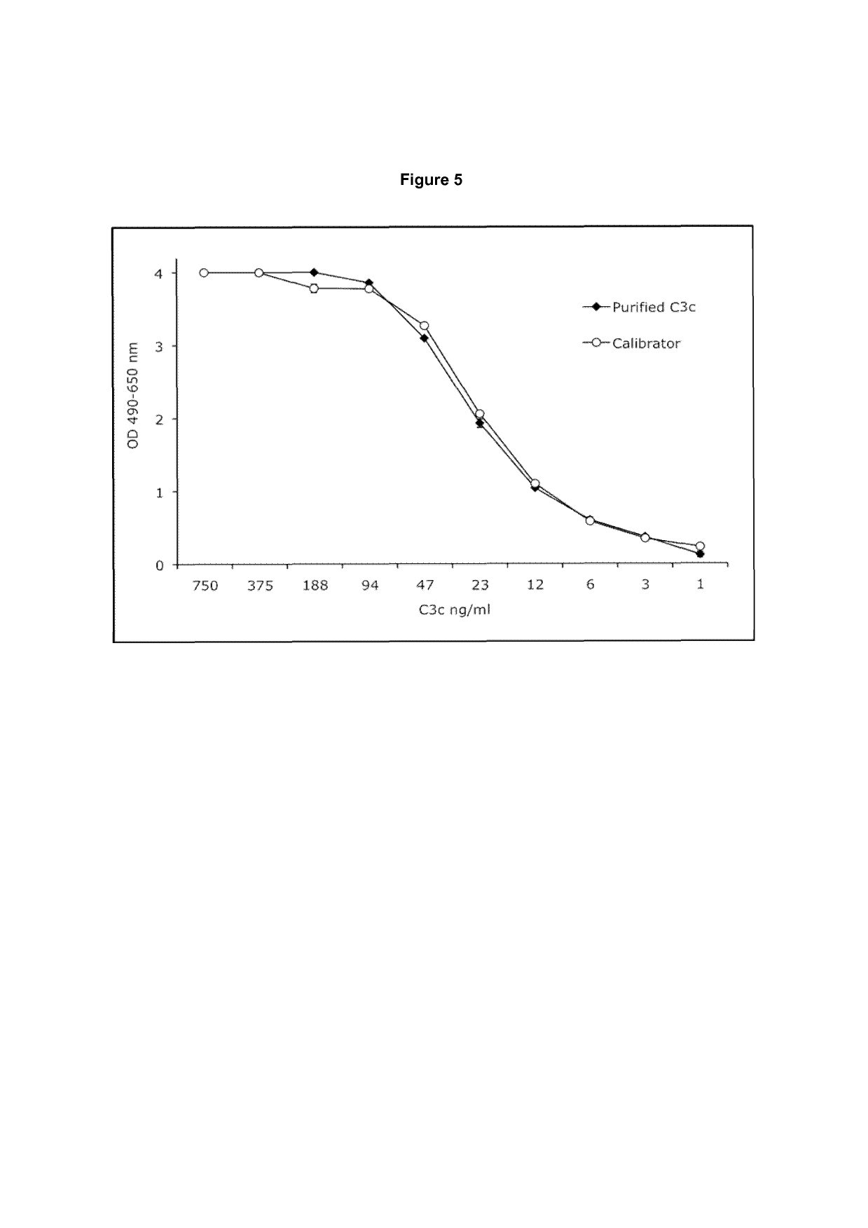

In FIG. 5 calibration curves and parallelism is shown. A calibrator made of a pool of normal plasma was analyzed in together with a preparation of purified C3c with a known content of C3c. Parallelism was validated by diluting the plasma pool calibrator against purified C3c and was adjusted to the best fit with the as OD_{490-650 nm}-units obtained from the calibrator.

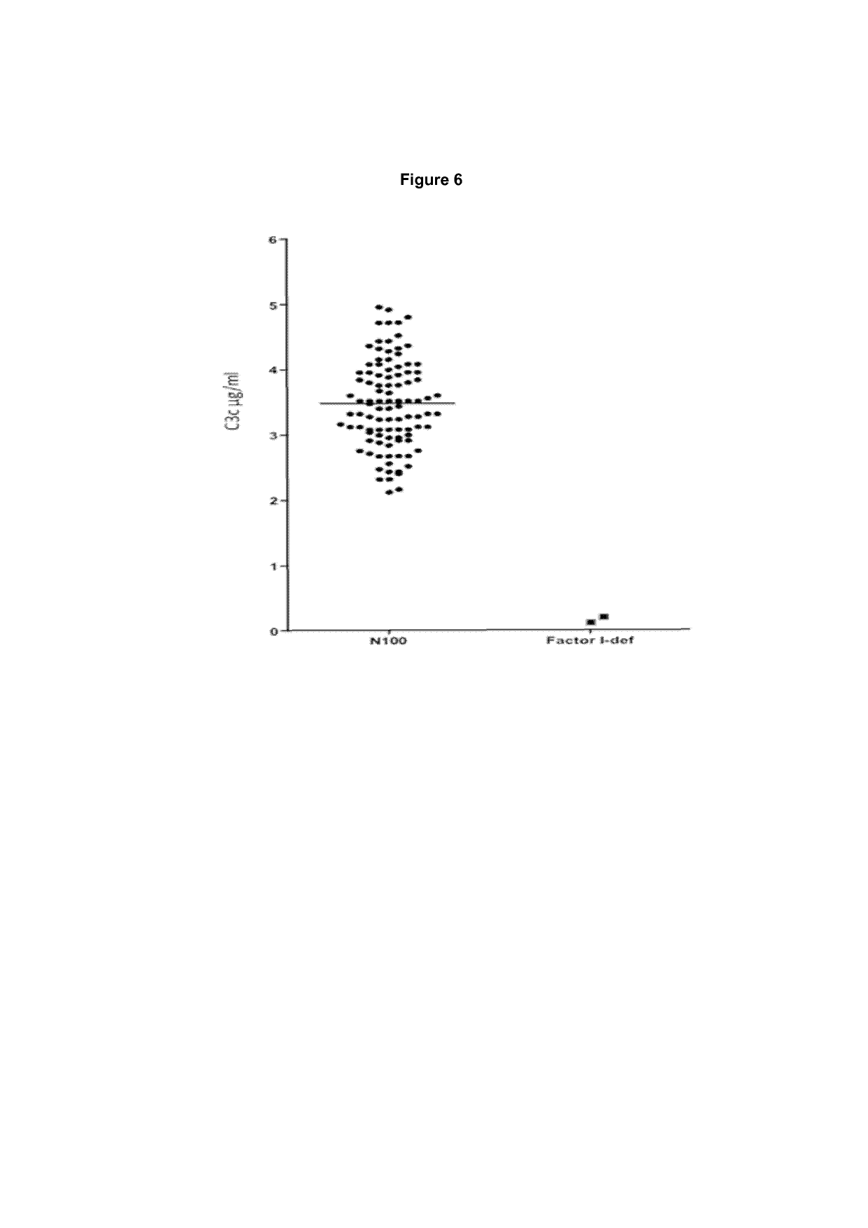

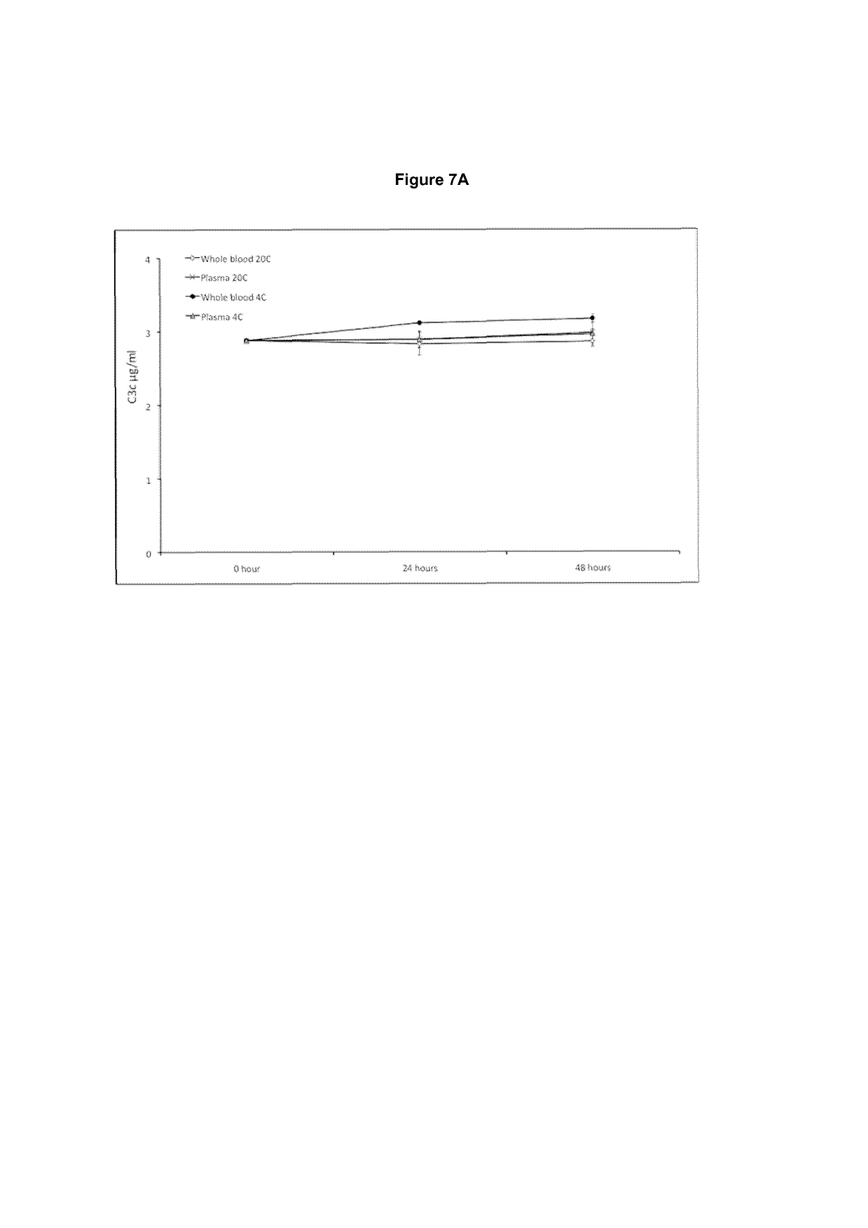

In FIG. 6 the C3c range in plasma from 100 Danish blood donors and the C3c level in plasma samples from two different factor I-deficient patients is shown. The solid lines indicate the mean value of the samples tested.

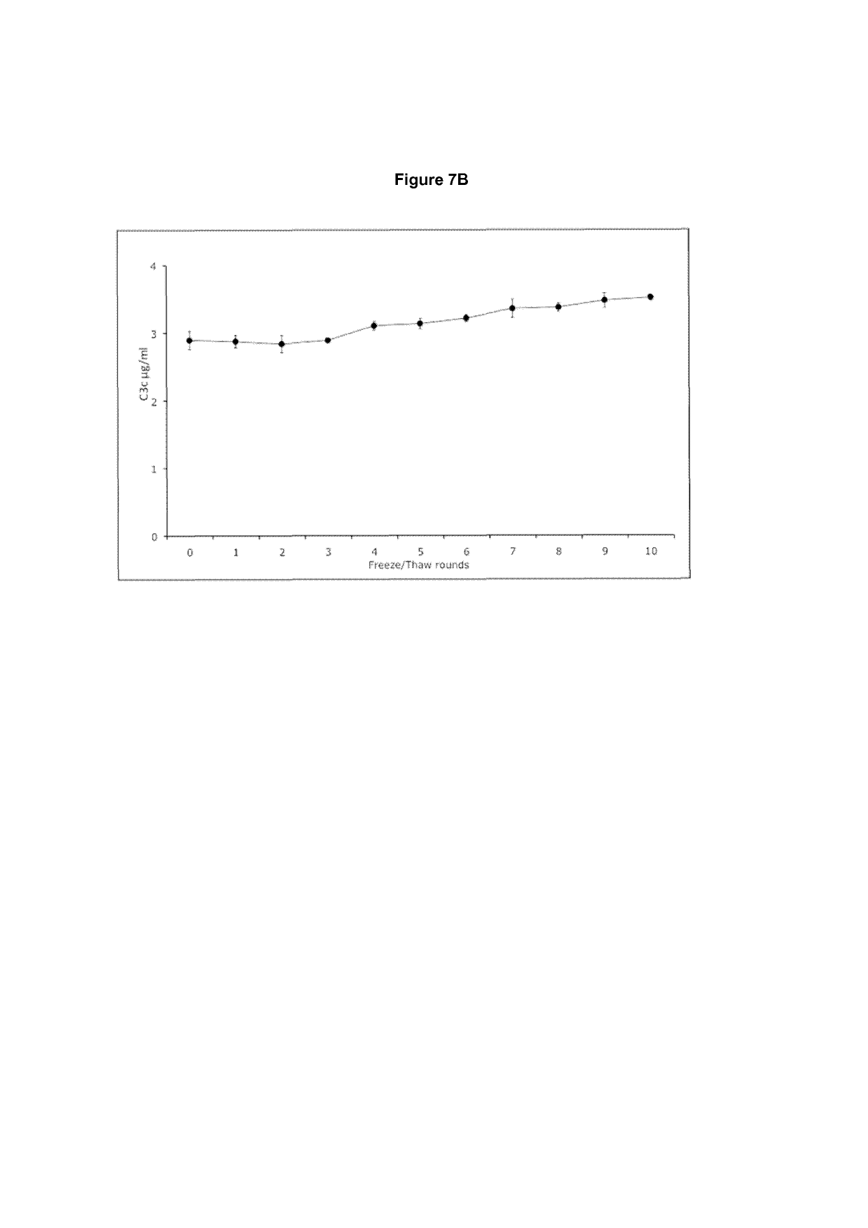

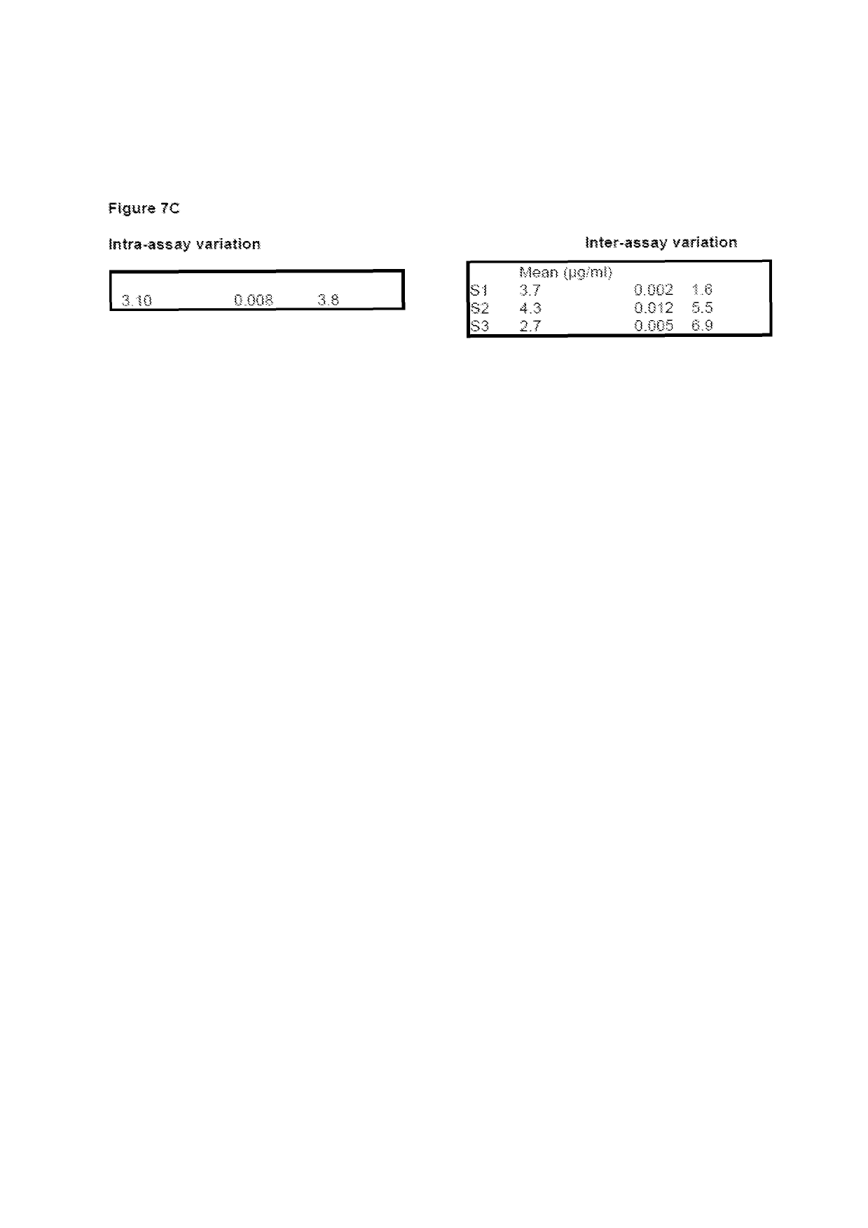

In FIG. 7A the influence of freeze and thawing is shown. EDTA plasma was frozen and thawed for 10 rounds and the C3c levels were measured for each round. In FIG. 7B the effect of storage is shown. Samples of either EDTA plasma or EDTA full blood was stored for up to 48 hours at 4 or 20° C., followed by assessment of the C3c level.

In FIG. 7C analysis of the intra-assay and inter-assay variation is shown. For the assessment of intra-assay variation of the C3c sandwich ELISA one sample was tested in 40 wells on one occasion. For the inter-assay variation, three samples were tested on six different occasions. The coefficient of variation (CV=SD/mean×100%) was calculated.

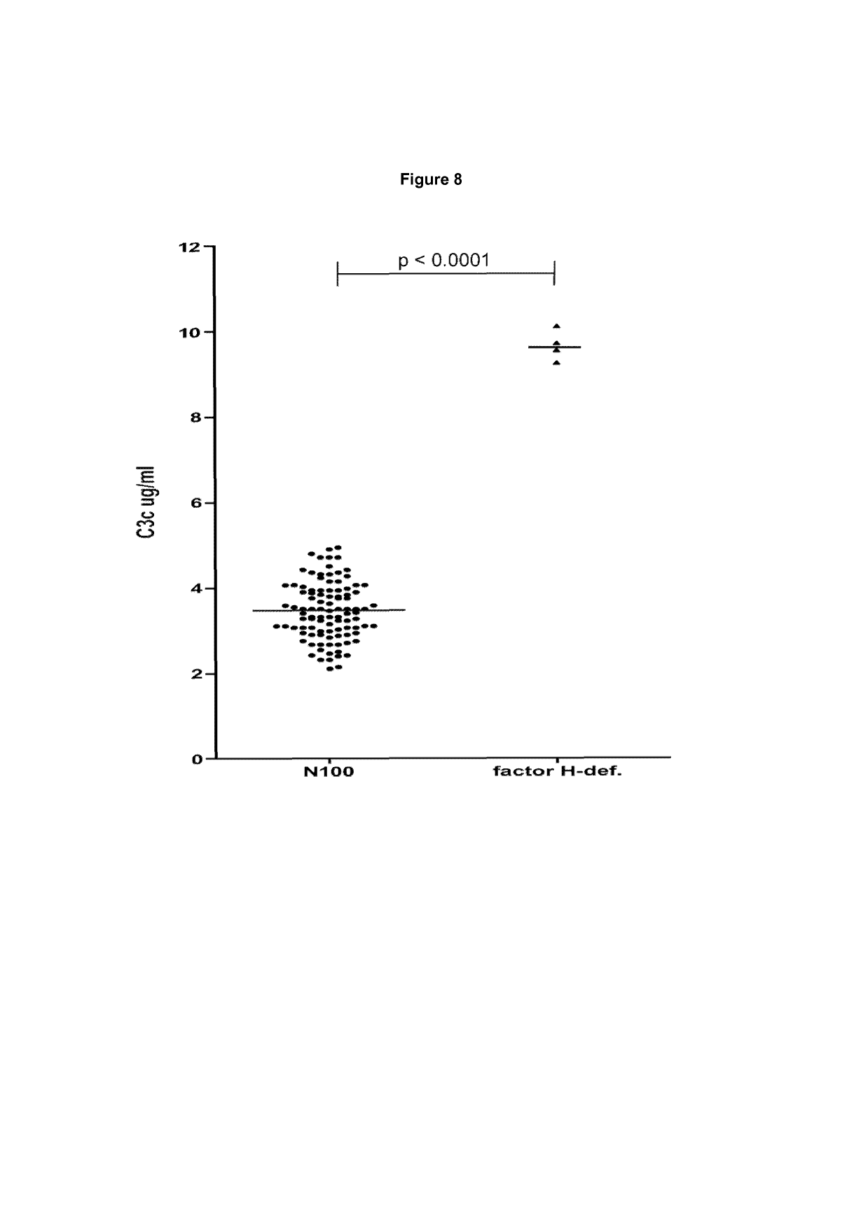

In FIG. 8 assessment of the C3c level in four factor H deficient patient with elevated complement activity and distribution range in 100 healthy blood donors is shown. Non-parametric test was used to test for significance.

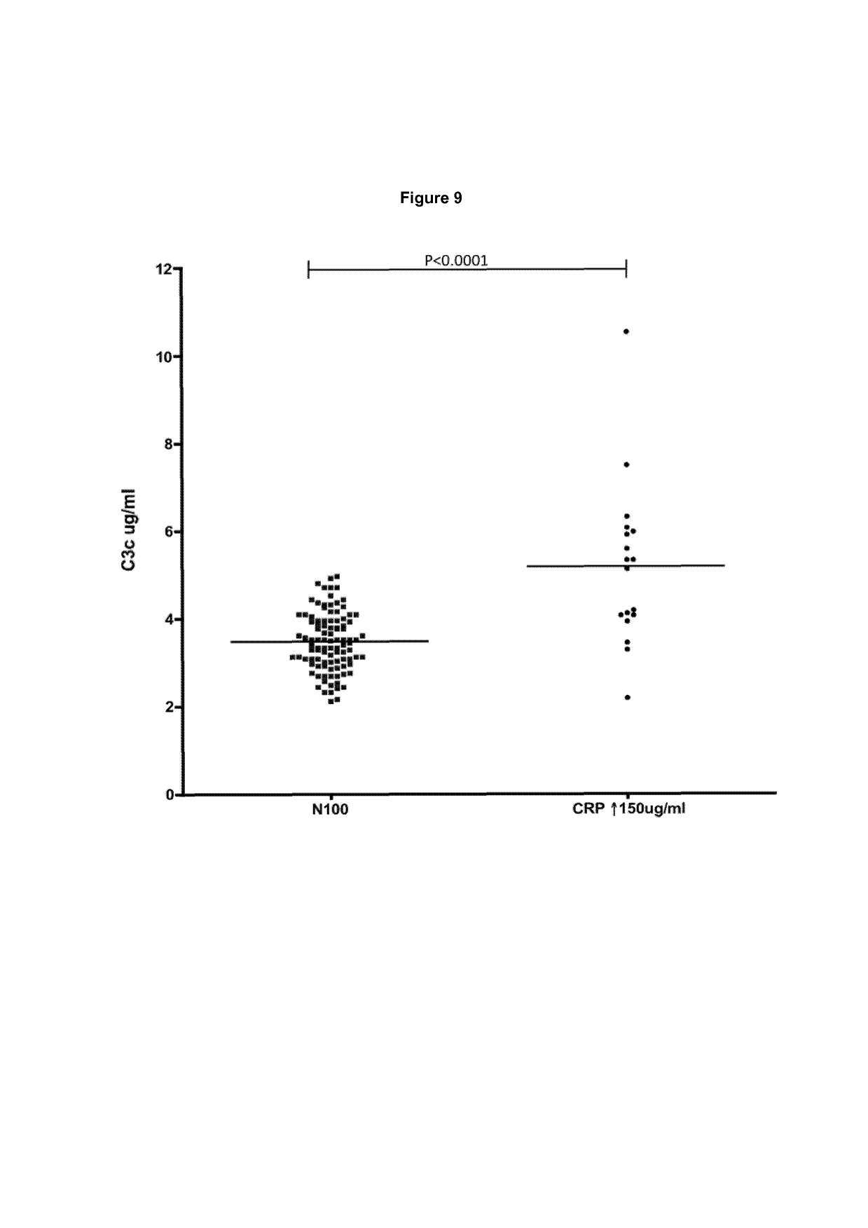

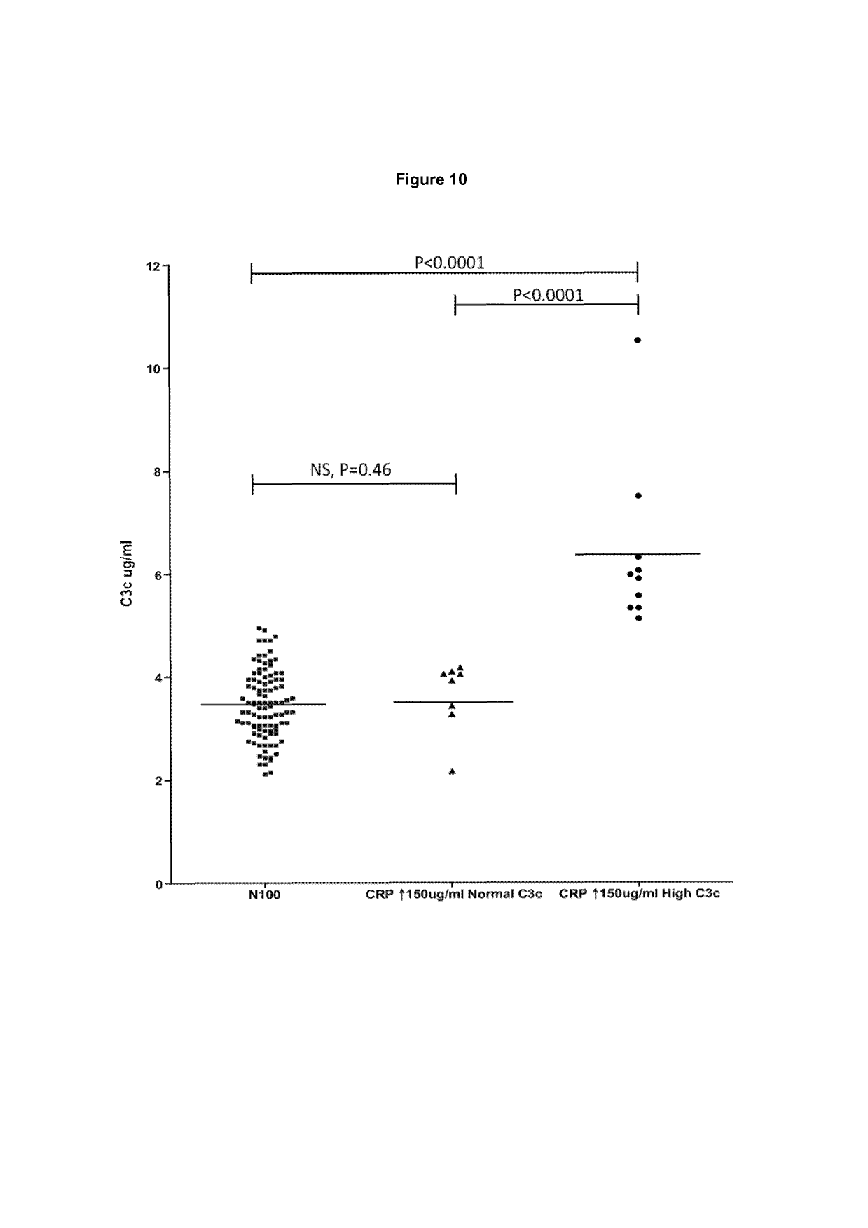

In FIGS. 9 and 10 assessment of the C3c level in 18 patients with elevated CRP and suspicion of a systemic infection is shown. Comparison with the distribution range of 100 healthy blood donors. Non-parametric t test was used to test for significance.

### MATERIALS AND METHODS

### Reagents and buffers

Rabbit anti-human serum albumin (A001), horseradish peroxidase (HRP) conjugated rabbit anti-mouse IgG (P0260), Rabbit anti-human C3c (A0062), Rabbit anti-human C3d

(A0063) and HRP conjugated streptavidin (P0397) were all from Dako, Denmark. Buffers for ELISA assays: Coating buffer (35mM NaHCO₃, 15mM Na₂CO₃, pH 9,6), staining buffer (35mM C₆H₈O₇, 65mM Na₃HPO₄, pH 5), washing buffers: PBS, 10mM EDTA, 0.05% Tween-20, pH 7,4), or: TBS, 4mM Ca, 2mM Mg 0.05% Tween-20, pH 7,4.

### Generation and Purification of C3c Monoclonal Antibodies

In collaboration with Immunobond ApS (Frederiksberg, Denmark) we have developed a monoclonal antibody against human C3c. BALB/c×NMRI mice were immunized subcutaneously three times with 25 μg of C3c antigen coupled onto PPD (a mycobacterium purified protein derivate, Statens Serum institute, Copenhagen) adsorbed to Al(OH)₃, mixed in 1:1 ratio with Freunds incomplete adjuvant. Four days prior to the fusion the mice received an intravenous injection with 25 μg of the antigen administered with adrenalin. The fusion and selection was done essentially as described by Köhler and Milstein [13]. The SP2/0-AG14 myeloma cell line was used as fusion partner. Positive clones were selected by differential screening against C3 derivatives from in vitro activated normal human serum or factor I deficient serum in ELISA. Cloning was performed by limited dilution. Single clones were grown in culture flasks in RPMI+10% FCS and mAbs were purified from culture supernatant by protein A affinity chromatography using the Akta FPLC system according to the manufacturer's instructions (Amersham Pharmacia, Uppsala, Sweden)

In vitro Activation of Serum Complement

Inulin (SIGMA®, 13754-25G), cobra venom factor (CVF, SIGMA®, No. V-9125) and aggregated human IgG (HA IgG, prepared from human IgG, for intravenous use, Statens Serum Institut, Denmark) were used as activators of serum complement. Aggregated human IgG was prepared by incubating human IgG (1 mg/ml) at 56° C. for 60 min. Large aggregates were removed by centrifugation at 1400×g for 5 min. Serum was incubated with 0.5 mg/ml of inulin, CVF or HAG for 37° C. for 60 min. After the activation samples were made 10 mM with respect to EDTA, centrifuged at 1400×g for 5 min and frozen at −80° C., awaiting further analysis.

### Immunoprecipitation of C3 Products

Immunoprecipitation of serum C3 was performed with the C3c specific anti human C3c mAb HuC3c F1-4 or an anti human C3 mAb HuC3 F1-8 (a monoclonal antibody reacting against the beta chain of C3/C3b/C3c) as a control. Additionally a mouse IgG antibody (IgG1K) with no known specificity was applied as a negative control. A total of 10 μg of mouse mAbs (HuC3c F1-4, HuC3 F1-8 or IgG1K) was allowed to bind to sheep anti mouse IgG Dynabeads (M-280, cat. 112.02D, Dynal/Invitrogen). After a washing step the beads were applied to 50% inulin activated normal human serum (NHS) (see above) and 50% of non-activated normal human EDTA plasma (NHP) and incubated end over end for 1 hour at 4° C. After the final washing steps and magnetic separation, the beads were boiled in SDS loading buffer and subjected to SDS-PAGE and immunoblotting probed with polyclonal antibodies to C3c and C3d (A0062, A0063, Dako).

### Immunoaffinity Purification of Serum C3 Products

Antibody affinity purification was used to purify C3c from serum. In brief 50 mg of anti C3c mAb f1-4 or anti C3 mAb 8 was covalently coupled to CNBr activated sepharose essentially as described by Pfeiffer et al. [14] and used as the purification matrix. 10 ml of in vitro activated NHS were applied to the matrix, which were subsequently washed with PBS. Fractions were eluted with 0.5% citric acid and analyzed by SDS-PAGE and coomassie staining or immunoblotting.

### SDS-PAGE and Immunoblotting

Electrophoresis was performed on 4-12% (w/v) polyacrylamide gradient gels using the NuPAGE□ system (Invitrogen) according to the manufactures recommendations. Proteins were electro blotted onto Polyvinylidene difluoride membranes (PVDF-HyBond, Amersham Bioscience). After blocking in PBS, 0.05% Tween20 membranes were incubated with 2 μg biotinylated polyclonal C3c, 0.05% Tween-20 followed by incubation HRP conjugated streptavidin (P0397) diluted 1:2000 in PBS, 0.05%-Tween20 at RT for 1 h. The membranes were washed and developed with 0.04% 3-amino-9-ethylcarbazole (Sigma) and 0.015% H202 in 50 mM sodium acetate buffer, pH 5.0.

### Assessment of Deposited C3 on Solid Phase Immune Complexes

The assay to measure deposited C3 on solid phase immune complexes was performed as described by Palarasah et al [15] using 2 μg of a mixture of biotinylated polyclonal anti C3c and polyclonal anti C3d or mAb HuC3c F1-4.

### C3c Specific Sandwich ELISA

The assay is constructed as a non-competitive indirect sandwich ELISA using mAb HuC3c F1-4 as capture antibody and biotinylated polyclonal anti human C3c and streptavidin conjugated HRP for detection. Maxisorb plates (Nunc, Roskilde, Denmark) were coated overnight at 4° C. with 100 pl of 6 pg/ml of mAb HuC3c F1-4. Wells were emptied, washed three times in washing buffer and incubated with serial dilutions of the calibrator (see below) or samples, both diluted in PBS, 10 mM EDTA, 0.05% tween 20 for 60 min at 20° C. Wells were emptied, washed three times with washing buffer and incubated for 45 min at 20° C. with 100 μl of 2 pg/ml of biotinylated polyclonal C3c in washing buffer. Wells were emptied, washed three times in washing buffer and incubated at 20° C. with 100 μl of streptavidin conjugated HRP (Zymed, Invitrogen) diluted 1:3000 for 30 min. The plates were developed as described previously [16].

### Analysis of Intra-Assay and Inter-Assay Variation

For the assessment of intra-assay variation of the C3c specific ELISA assay, one sample was tested in 20 wells on one occasion. For calculation of the inter-assay variation, three samples were selected and tested on six different occasions. The mean values, standard deviation (SD) and the coefficient of variation (CV=SD/mean×100%) were calculated for these ELISA setups.

### In Silico Analysis

Determination of C3c concentrations were based on fitted standard curves using optical density values obtained by serial dilutions of a plasma pool from 100 healthy blood donors, by means of the software Softmax Pro® (Molecular Devices, Calif., USA). Linear regression, statistics (regression analysis, non-parametric two-tailed t-test) were calculated using Prism4 software (GraphPad Software, Inc.).

### RESULTS

Immunoprecipitation of C3 products

The specificity of the generated C3 antibodies was evaluated with different techniques. The putative C3c specific HuC3c mAb4 and a characterized anti C3 mAb 8 reacting with the R-chain of C3 were used to precipitate C3 products from in vitro activated NHS (mixed half'n'half with NHP). The precipitates were subsequently analyzed by non-reduced SDS-PAGE and immunoblotting probed with polyclonal antibodies to C3. When serum C3 was precipitated with mAb 8 the inventors observed bands with apparent molecular mass of 180 kDa, 175 kDa and 140 kDa corresponding to native C3, C3b/iC3b and C3c respectively (FIG. 1). In contrast to this the HuC3c mAb4 only precipitated one distinct band with an apparent molecular mass of 140 kDa corresponding to the intact C3c fragment. No bands were evident when the inventors used an IgG1K as a precipitation isotype control.

### Immunoaffinity Purification of Serum C3

Based on the immunoprecipitation results, we used the putative C3c specific mAb HuC3c F1-4 and the anti C3 mAb HuC3 F1-8 for column immunoaffinity purification (IAP). We used CVF activated NHS mixed half'n'half with NHP as the C3 source for the purification. The IAP eluted fractions were subsequently analyzed by SDS-PAGE under non-reducing and reducing conditions and stained with coomassie directly or immunoblotted using polyclonal anti C3c and C3d antibodies as detection antibodies. Eluted fractions from the mAb HuC3c F1-4 column produced a distinct band with apparent molecular mass of 140 kDa in non-reducing SDS-PAGE and coomassie staining or immunoblotting corresponding to the intact C3c molecule (FIG. 2, left side). The same fractions analyzed under reducing conditions followed by coomassie staining revealed bands with apparent molecular mass of 75 kDa, 40 kDa and 25 kDa (FIG. 2, right side). These bands correspond to the R-chain, the α2 and α1 domains of C3c, respectively. The β-chain and α2 of C3c were also evident in the corresponding immunoblot, whereas the missing α1 is caused by a total absent reaction in the polyclonal anti C3 antibody (data not shown). The eluted fractions from the Ab HuC3 F1-8 IAP produced bands of 180 kDa, 175 kDa and 140 kDa corresponding to native C3, C3b/iC3b and C3c under non-reducing conditions (FIG. 2 left side). Prominent bands corresponding to intact a- and R-chain (110 and 75 kDa, respectively) were evident under reducing conditions/coomassie staining (FIG. 2, right side), but weaker bands from α2 and α1 were also observed. This pattern was also evident in the corresponding immunoblot except for the α1 band.

### C3 Deposition On Solid Phase Immune Complexes

Another experiment to verify the specificity of the mAb HuC3c F1-4 was carried out on deposited C3 derivates on solid phase immune complexes. Dilutions of NHS were incubated on an immune-complex surface (ICS). Activation and deposition of C3b, iC3b and C3d were assessed using mAb HuC3c F1-4 or a mixture of polyclonal anti-human C3c and C3d antibodies. The mAb HuC3c F1-4 showed no reactivity with deposited C3 derivatives on solid phase immune complexes i.e. C3b, iC3b and C3d (FIG. 3). In contrast a significant dose dependent binding curve was observed with the polyclonal anti C3 antibodies on the ICS (this was also evident with the mAb HuC3 F1-8, data not shown). When the supernatants from the ICS activation were analyzed using the mAb HuC3c F1-4 as catching antibody we measured a very significant dose dependent curve of C3c and a major increase in the signal compared with the EDTA ICS incubated NHS (FIG. 3). The inventors observed the same phenomena when we analyzed the reactivity pattern of the HuC3c mAb4 to deposited C3 on other complement activating surfaces such as lipopolysaccarides (alternative pathway) and mannan (lectin pathway) (data not shown).

### Specific Antigen Reactivity/Elucidation of the Epitope

To further define the fine specificity of the mAb HuC3c F1-4 we synthesized the C-terminal part of the alpha 1 chain of C3c with the C-terminal end residue arginine with the native carboxy terminal end or with an amidated C-terminal end. We also synthesized a larger peptide of C3 covering the factor I cleavage site between the C-terminal part of alpha 1 and the N-terminal part of C3d. The reactivity of mAb HuC3c F1-4 was exclusively against the peptide with the native carboxy terminal end and showed no reactivity against the amidated peptide as well as the peptide bridging the C3c alpha 1 and C3d part. This thus shows that the antibody specificity is dependent on the natural carboxy terminal end of C3c alpha 1, which is only exposed after factor I cleavage.

Thus, taken together these findings show that the generated HuC3c mAb4 has exclusive specificity for C3c and does not cross-react with other C3 derivates.

### C3c Sandwich ELISA

The HuC3c mAb4 was used to develop a non-competitive indirect sandwich ELISA. The setup was based on the HuC3c mAb4 as a capture antibody and biotinylated polyclonal anti C3c as the detection antibody. The inventors used this setup to analyze in vitro activated serum (activated with Inulin, CVF or HAG), factor I deficient plasma and plasma from healthy blood donors. Analysis of the activated sera resulted in dose dependent titration curves of C3c. In the activated samples the inventors were able to detect C3c in dilutions up to 1:40290 (FIG. 4). The pool of normal human plasma also showed a nice dose dependent titration curve of C3c, which represent the basal level of C3c in none activated plasma (FIG. 4). In addition the inventors analyzed plasma from two well characterized factor I deficient patients as negative controls [17]. Due to the lack of functional factor I these patients are not able to cleave and degrade C3b and the inventors could not measure C3c above background level (FIG. 4).

### C3c Plasma Levels

The developed quantitative sandwich ELISA was used to determine the distribution range of C3c level from 100 healthy Danish blood donors. In order to calibrate the ELISA in absolute concentrations, a serial dilution of a pool of normal plasma served as calibrator and was compared to serial dilutions of purified C3c. The inventors observed perfect parallelism between the calibrating standard curve and the dilution curves of purified C3c and the inventors determined the concentration of plasma pool to be 4.01 μg/ml (FIG. 5). This plasma pool was stored according to the seed-lot system and served as calibrator to determine the concentration of 100 healthy plasma samples. The inventors found a mean C3c level of 3.47 μg/ml and a range of 2.12-4.92 μg/ml (FIG. 6). In addition the inventors analyzed plasma from two well defined factor I deficient patients, which was below 0.2 μg/ml. The factor I deficient patients have previously been described by factor I antigen absence by crossed immunoelectrophoresis (XIE). We assessed the amount of factor I by specific monoclonal antibodies to factor I in a two side ELISA and found the factor I antigen amount to be 2.5% of the normal range in the first patient (resulting in 0.2 μg/ml of C3c antigen) and 0.6% in the second patient (resulting in 0.1 μg/ml of C3c antigen) (data not shown). The presence of residual factor I explains the small amount of cleaved C3c antigen present in the sampled from the deficient patients.

The inventors also evaluated the influence of EDTA plasma and whole blood held at 4 and 20° C. for 0-48 hours and repetitive freeze/thaw cycles of EDTA plasma. Storage for up to 48 hours at 4 or 20° C. of either EDTA full blood or plasma did not lead to significant in vitro activation of complement and a sub sequential C3c levels (FIG. 7A). The level of C3c was constant in EDTA plasma, which has been repetitive frozen and thawed up to four times where after a slight increase was observed (FIG. 7B). The assay variation is shown in FIG. 7C. The intra-assay variation was 3.8% where one sample was tested in 40 wells on one occasion. The inter-assay variation was below or equal to 6.9% where three different samples were tested on six different occasions. The coefficient of variation: CV=SD/mean×100%.

Patient Samples with Expected Systemic Inflammation

Factor H deficiency is associated with increased ongoing complement activation. Samples from four factor H deficient patients were assessed and compared to the normal C3c range. A significant (p<0.0001) higher level of C3c was observed in this defined patient group compared to the normal range (FIG. 8).

Samples from another group of patients admitted to a hospital with suspected systemic infection and selected with high CRP levels were also evaluated for the C3c level. Significant (p<0.0001) higher level of C3c was observed in this group of 18 patients (FIG. 9), however the group can be subdivided into two groups of almost identical size; one which falls within the normal range and the other with a very high C3c level (FIG. 10).

### DISCUSSION

Markers for systemic inflammation in acute or chronic diseases are highly needed and factors of the complement system are obvious targets. This paper is the first, to our knowledge that describe an ELISA based method for the assessment of plasma C3c without interference from other complement products.

The inventors have developed a C3c specific monoclonal antibody that reacts against an epitope only exposed on the C3c fragment. Immunoprecitation of C3 products from serum demonstrated that the HuC3c mAb4 only precipitated one distinct band corresponding to the molecular mass of C3c. The specificity was further confirmed when the antibody was used to column affinity purify C3c from serum. The reduced and non-reduced eluted fractions from the affinity purification corresponded perfectly to the molecular parts of C3c. No fragments corresponding to C3, C3b, iC3b, C3a or C3d were observed. Purified C3c from serum was subsequently used to establish a plasma calibrator. The inventors also extended the analysis of the specificity of HuC3c mAb4 to deposited C3 products on solid phase activation matrixes. Incubation of serum samples on activating surfaces like immune complexes will result in deposition of complement products. Thus, split products of C3 such as C3b, iC3b and C3d will be represent on such solid phase surfaces whereas the generated C3c (which lack the thioester moiety) would gradually appear in the fluid phase. No reactivity of the HuC3c mA4 was observed against deposited C3 split products on the solid phase surfaces. In contrast to this the inventors could measure a significant rise of C3c in the corresponding fluid phase samples. Taken together these experiments demonstrate that the generated HuC3c mAb4 only reacts with C3c and does not cross-react with other C3 products.

A major problem associated with existing commercially available C3c antibodies is that these antibodies react both with C3c as well as with the C3c part of native C3 and iC3b/C3b. This will give rise to misleading picture of the C3c level in a given sample. Based on the C3c specificity of the HuC3c mAb4 antibody the inventors developed a non-competitive indirect sandwich ELISA to be able to give an accurate assessment of the plasma C3c level. The inventors observed perfect parallelism between purified C3c and the plasma calibrator demonstrating that the present ELISA setup can be used to measure the plasma concentration of C3c. The inter- and intra-assay variations were acceptable (<6.9% and <3.8%, respectively) and the inventors found that the plasma samples could be frozen and thawed for up to four times without any affects. The inventors presume that a small degree of ongoing in vitro activation even in EDTA plasma might be responsible for the slightly elevated level of C3c in repetitive freeze/thaw rounds (above four), as described previously for C3a [18]. Thus, the inventors conclude that repeated freezing and thawing for more that four times should be avoided.

In order to determine the plasma concentration range of C3c in healthy Danish blood donors the inventors analyzed 100 donor plasma samples and found a mean C3c level of 3.47 μg/ml and a range of 2.12-4.92 μg/ml. Very low levels of C3c level was observed in plasma samples from two different factor I deficient patients (a male and a female), which corresponded to the residual factor I antigen present in plasma samples (2.5% and 0.6%).

A recent study using in-gel digestion and mass spectrometry techniques in patients with neurodegenerative diseases and lung cancer showed that C3c might potentially be a useful biomarker in the pathogenesis of acute coronary syndrome, amyotrophic lateral sclerosis and parkinson's disease. [19, 20]. Even though C-Reactive Protein (CRP) is an important inflammatory marker other biomarkers like C3c have been suggested to be taken into careful consideration for inclusion in risk assessment algorithms [21]. The inventors therefore believe that the established C3c ELISA provides a simple method to acquire a detailed description of the ongoing complement activation and will therefore be invaluable in diagnosis, assessment of disease activity (treatment effect) and as a prognostic indicator.

In conclusion the inventors present a novel assay based on a well-characterized mAb that is able to accurately measure plasma C3c. The assay could potentially be of value in the assessment of the status during acute or chronic inflammatory processes.

### EXAMPLE 2

Based on the experimental procedures described below under items 1 and 2 this example shows how the C3c plasma level can be used to diagnose various disorders.

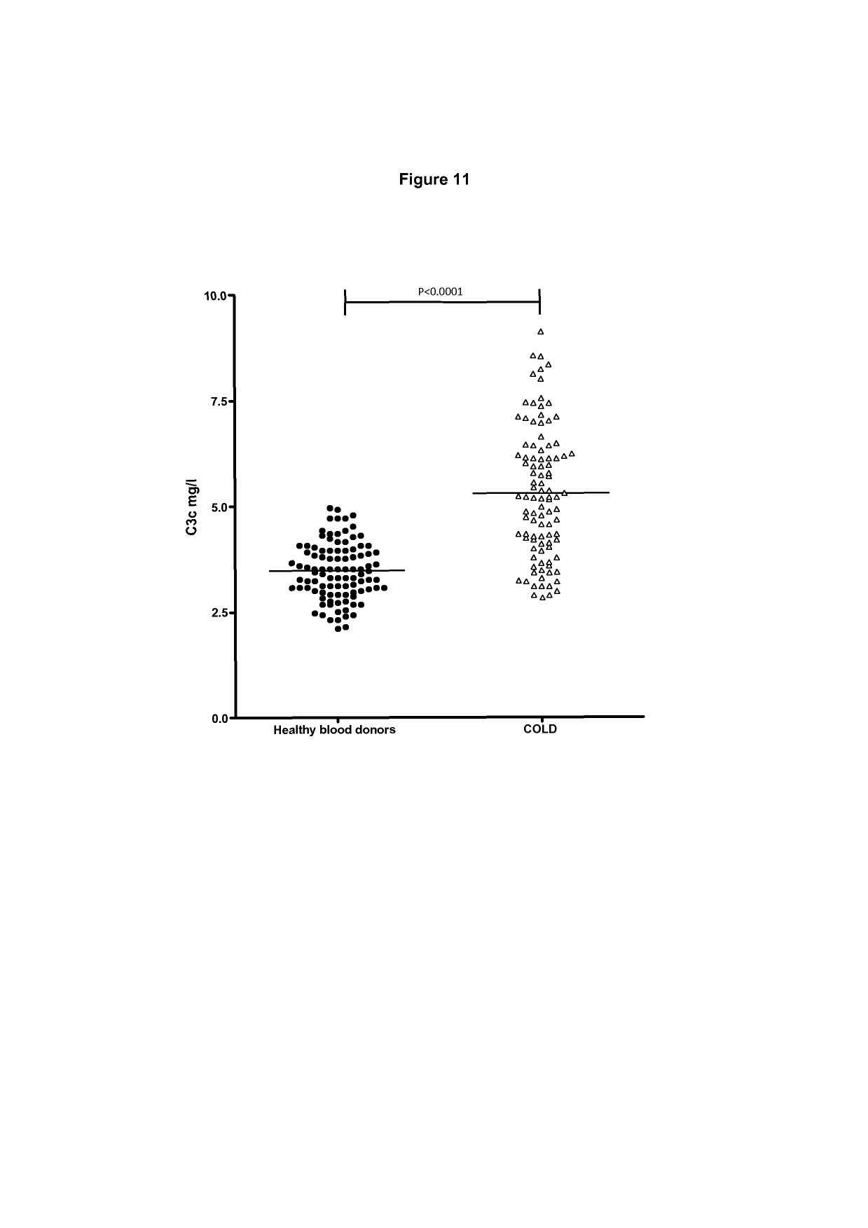

In FIG. 11 C3c plasma levels in 100 healthy Danish blood donors (left panel) and 100 Danish patients with chronic obstructive lung disease (COLD) (right panel) are shown.

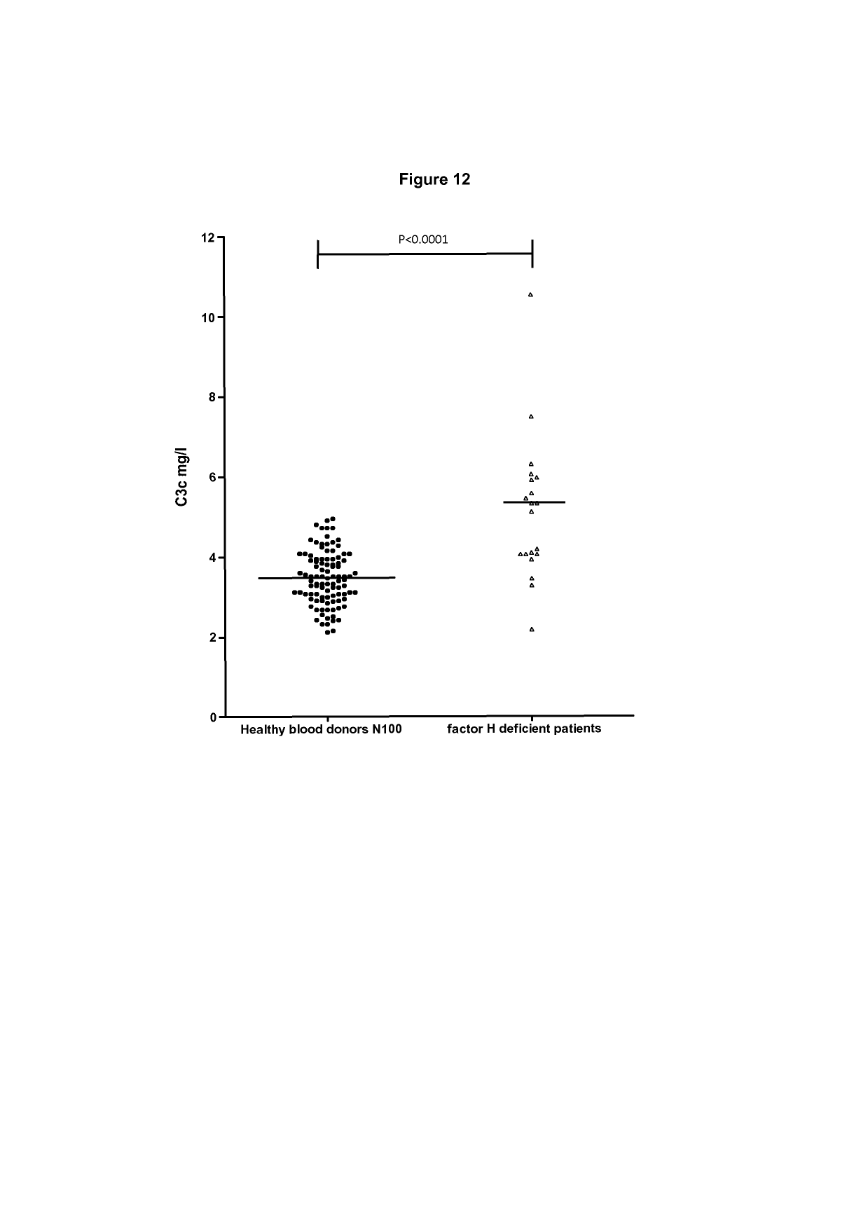

In FIG. 12 C3c plasma levels in 100 healthy Danish blood donors (left panel) and 20 patients with functional factor H deficiency (right panel) are shown.

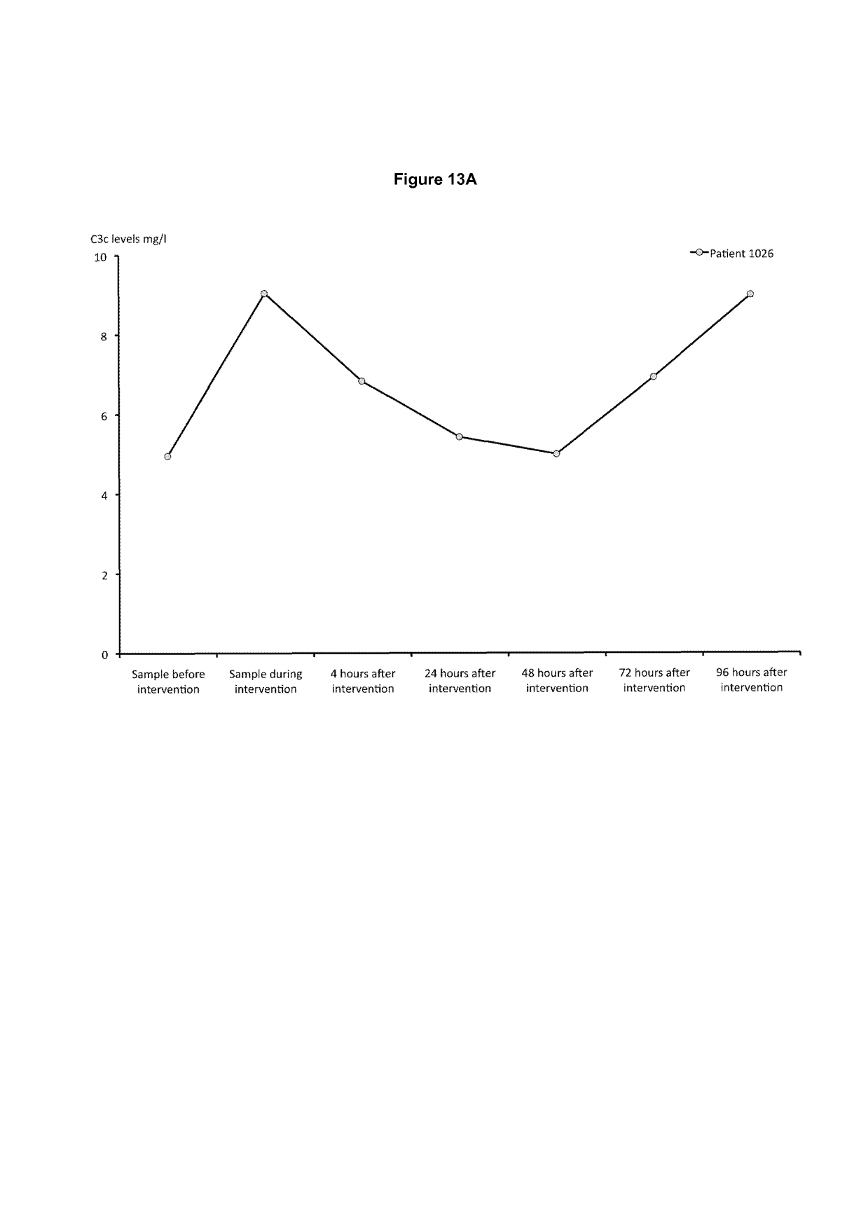

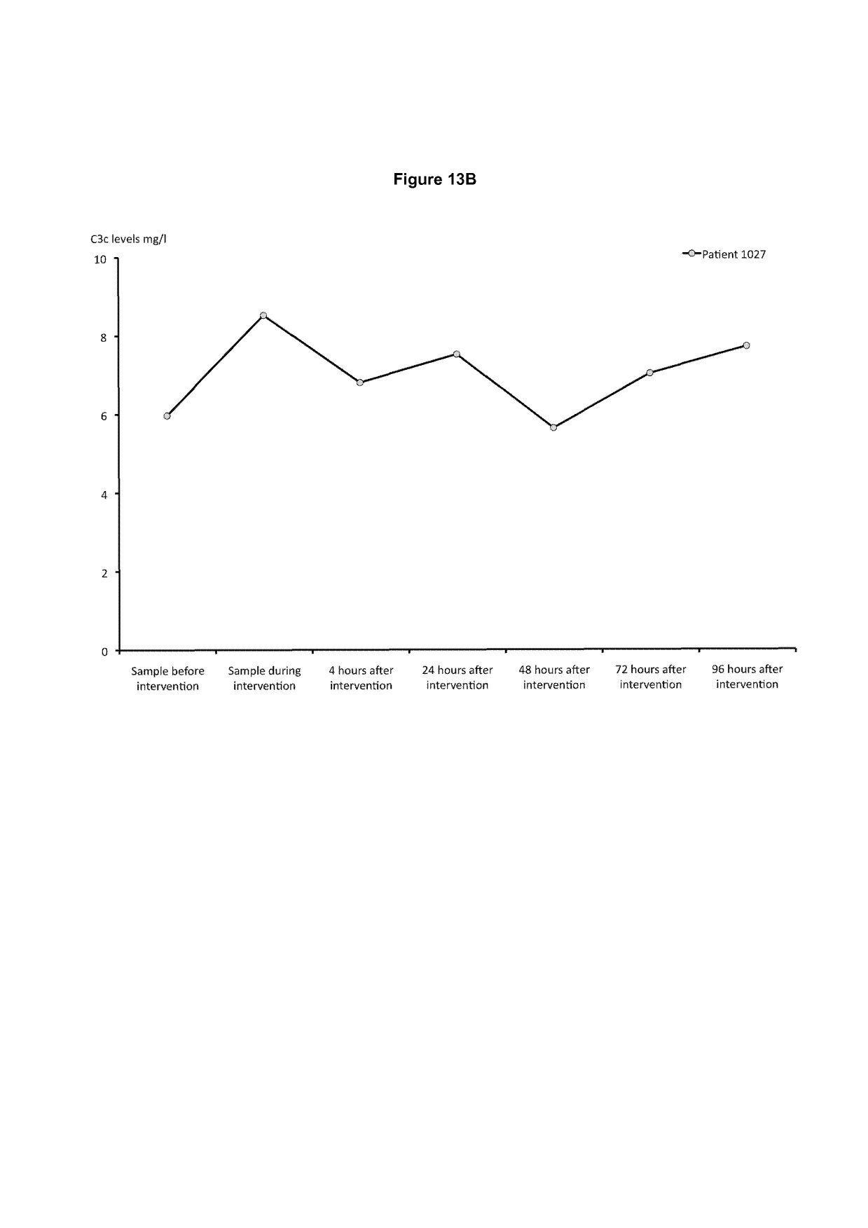

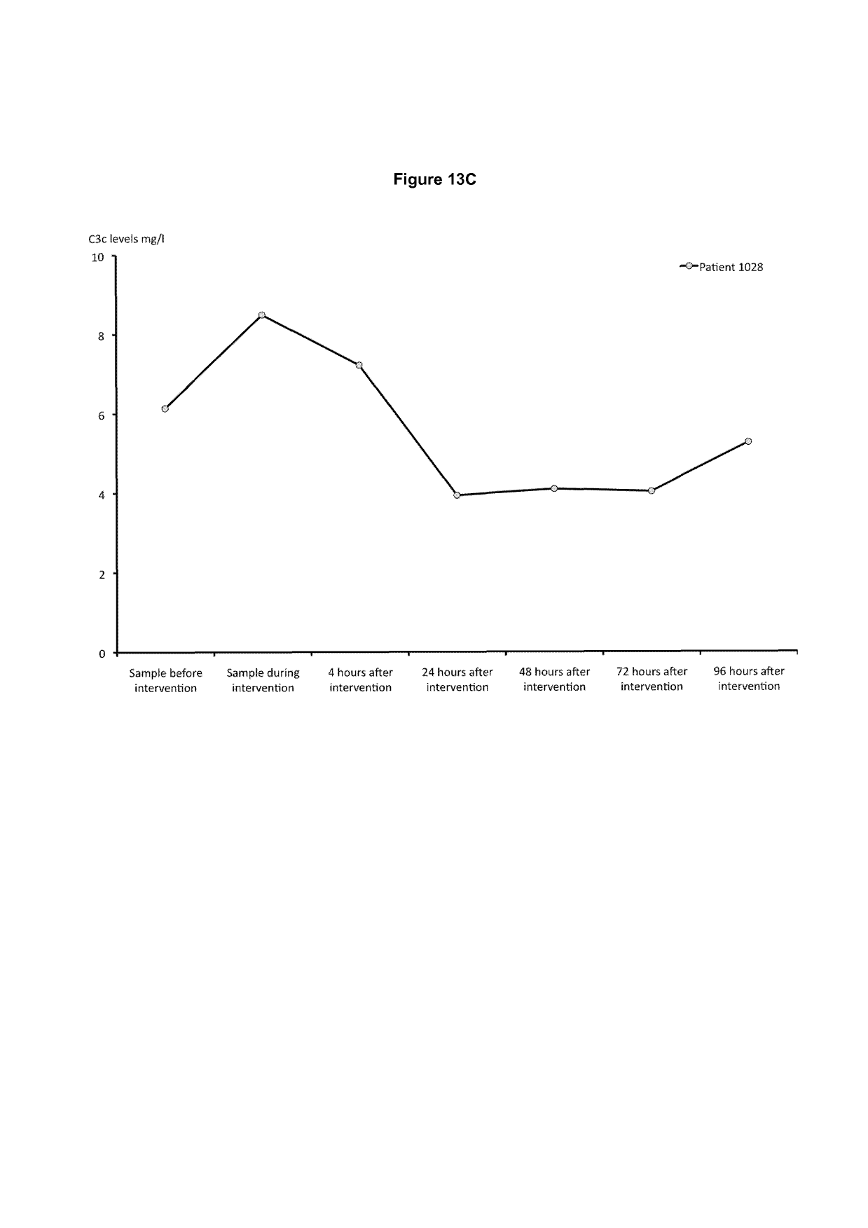

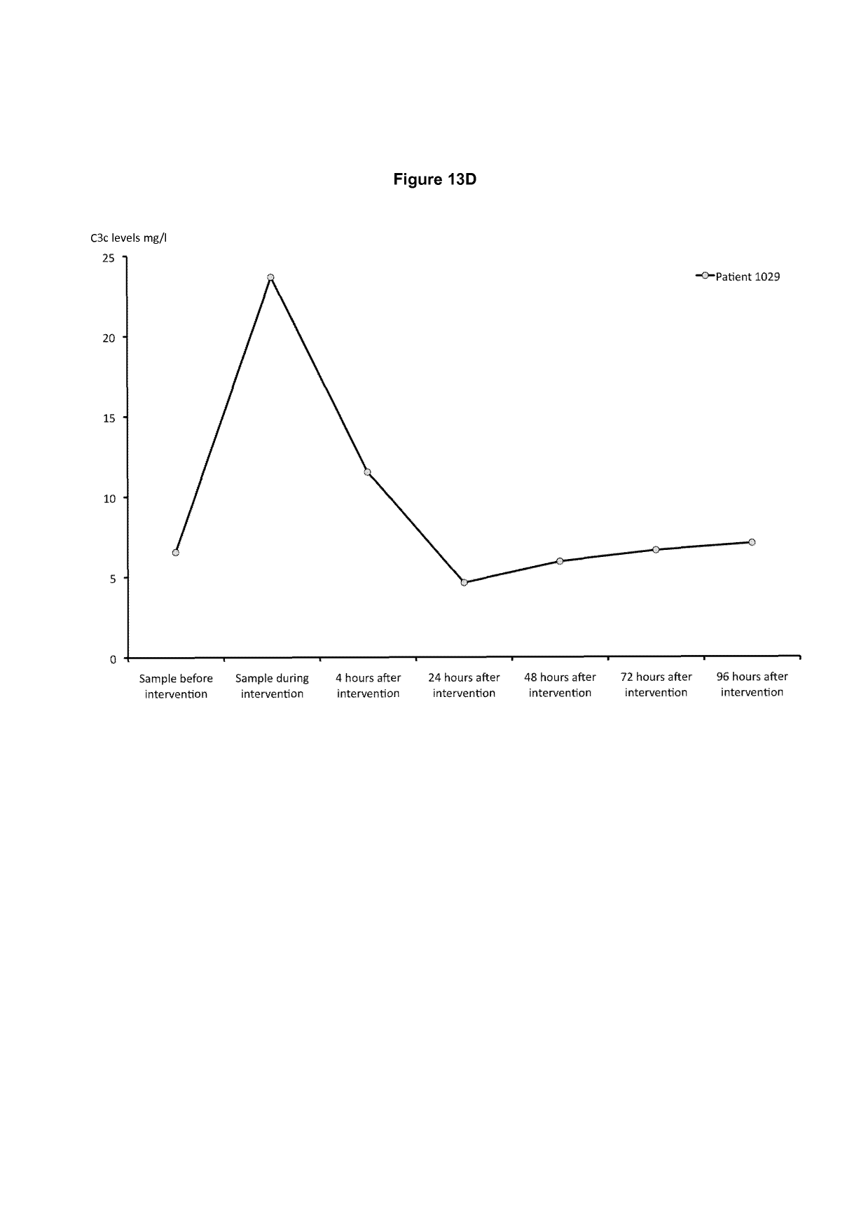

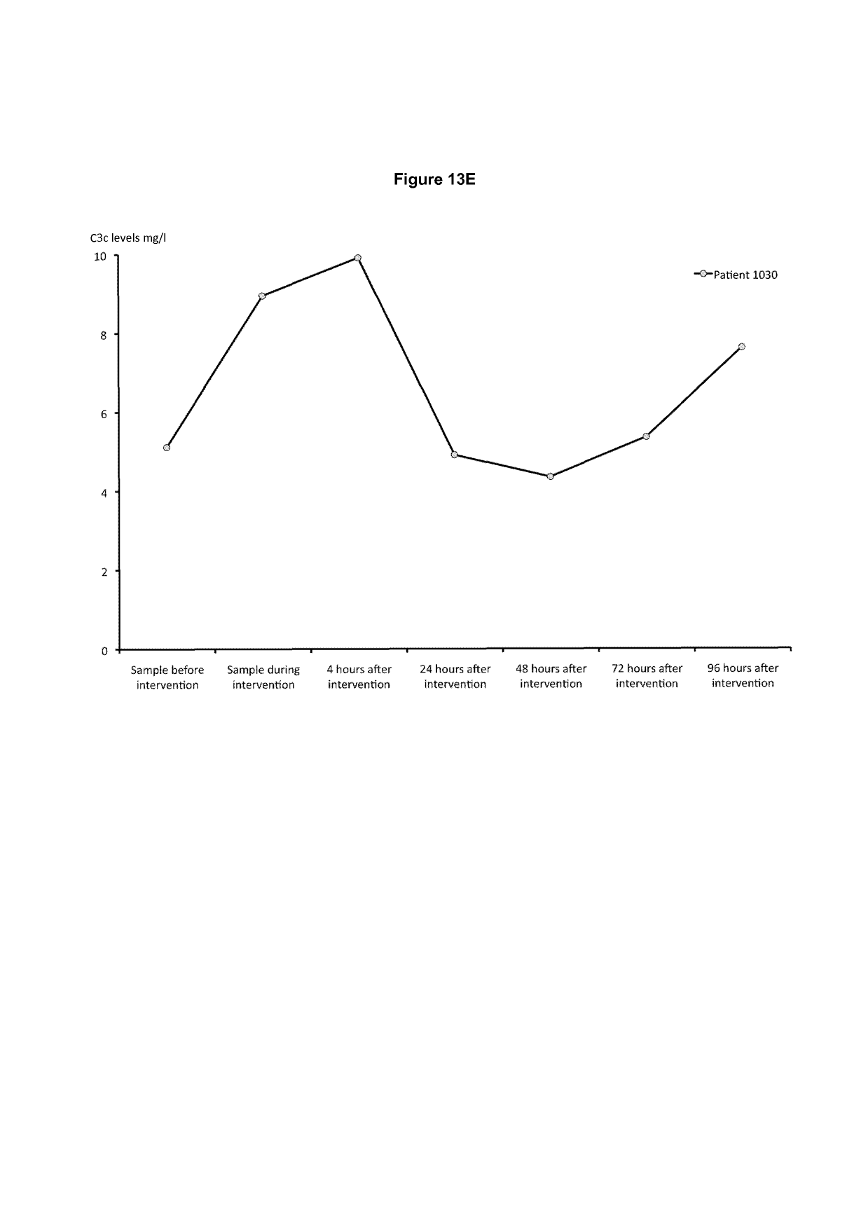

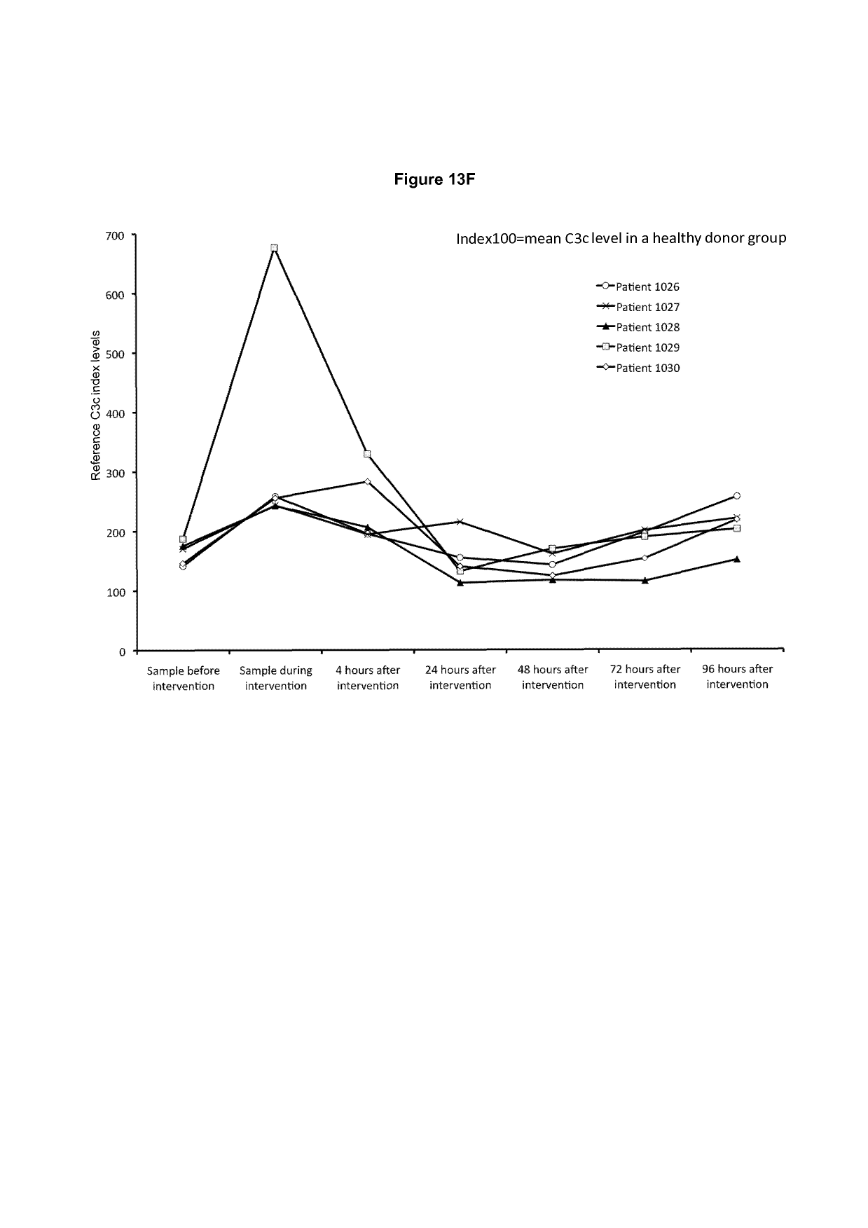

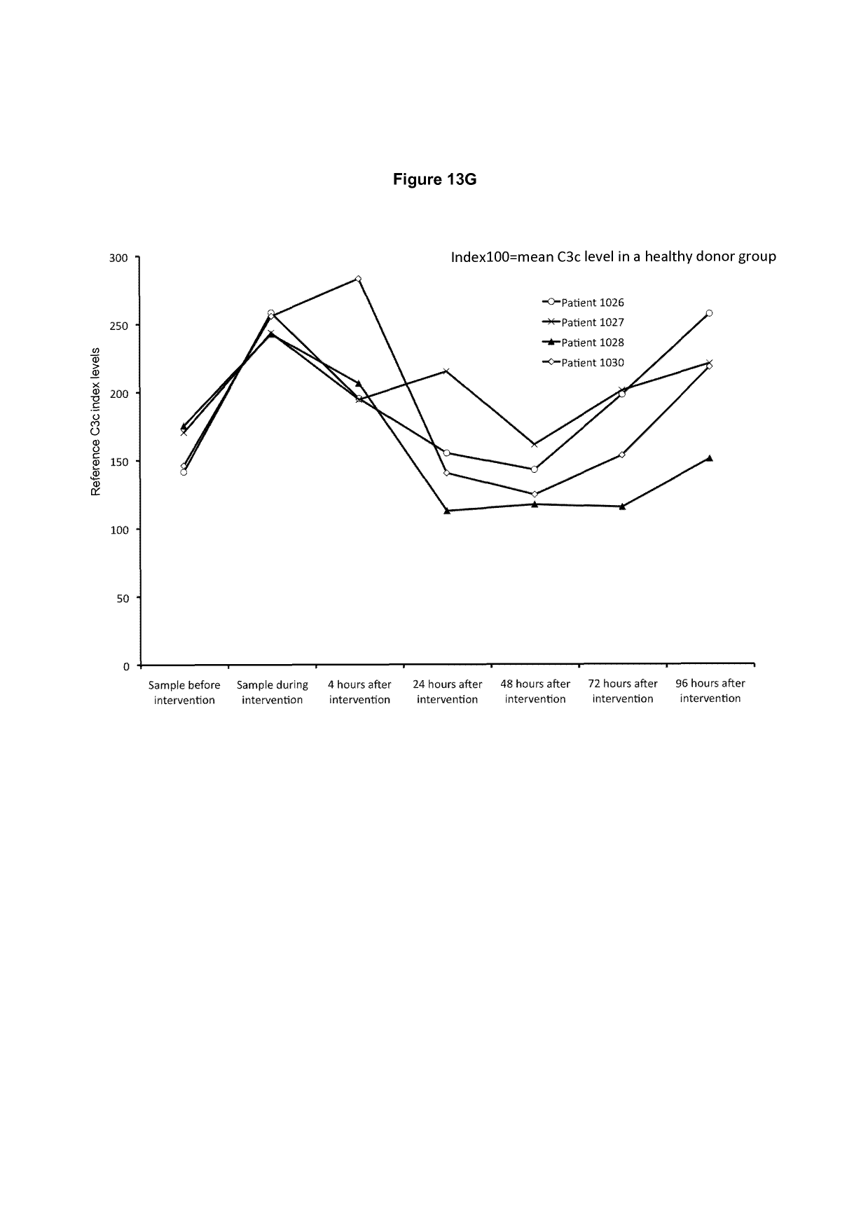

FIGS. 13A-E show: Plasma C3c levels in five patients admitted to the Department of Vascular Surgery, undergoing endovascular surgery. Samples are drawn before intervention, during intervention and 4 hours, 24 hours, 48 hours, 72 hours and 96 hours after intervention. FIGS. 13F-G show: The same five patient as above collected in two figures and related to the mean C3c level measured in healthy blood donors=lndex100 (3F includes patient 1029 that experiences an very high rise in the C3c level following intervention, 3G does not include patient 1029).

### 1.1 Sampling and Methods

Biological samples are EDTA plasma samples drawn in the same way according to the seed-lot system. After separation of the plasma by centrifugation the plasma samples are stored at −80° C. The C3c levels in the samples are measured as described previously (Yaseelan P et. al, J. Immun. Methods. 2010).

Statistical analysis (non-parametric two-tailed t-test, Mann-Whitney) was performed with the Prism4 software (Graph Pad Software Inc.).

### 2.1 Patient Samples

### 2.2 Chronic Inflammation

### Chronic Obstructive Lung Disease (COLD)

Background: Chronic obstructive lung disease (COLD) is a chronic lung disorder, which is characterized by low airflow in the lungs and decreased lung function, which may be caused by smoking, pollution or other noxious particles. The severity of COLD is dependent on a number of different factors but the disease is generally characterized by an abnormal and chronic inflammatory response in the lungs.

Results: A Danish cohort of patients (N100) with chronic obstructive lung disease (COLD) was measured for plasma C3c levels. This cohort represents patients in a chronic inflammatory phase. The C3c levels from the COLD patient samples were compared with plasma samples from a group of healthy Danish blood donors (N100). We measured significant higher levels of C3c in the COLD cohort compared to the blood donor group (FIG. 11) suggesting that chronic inflammation results in higher turnover of complement and generation of plasma C3c.

### Factor H Deficiency

Background: Functional factor H deficiency is characterized by a dysfunctional regulation of the complement system and especially by the regulation and turnover of complement factor C3 (and subsequent subcomponents, C3b, C3c and C3d). The symptoms that follow factor H deficiency are greatly dependent on the level of regulatory dysfunction but in general the patients will have a chronic systemic inflammatory state.

Results: A group of patients (N100) with functional factor H deficiency was measured for plasma C3c levels and compared with plasma samples from a group of healthy Danish blood donors (N100). We measured significant higher levels of C3c in the factor H patients compared to the blood donor group (FIG. 12).

### 2.3 Acute Inflammation

### Patients Undergoing Endovascular Aortic Repair

Background: Patients admitted to the Department of Vascular Surgery, Rigshospitalet/University hospital of Copenhagen, with asymptomatic aortic aneurysms. The patients undergo endovascular surgery and receive a stent graft in the aorta. The Ischemia-reperfusion injuries that follow vascular surgery mediates strong acute and systemic inflammatory responses

Results: A group of patients (N5) undergoing endovascular aortic surgery is followed and plasma samples are drawn:
1. Before intervention
2. When the intervention occurs
3. 4 hours after intervention
4. 24 hours after intervention
5. 48 hours after intervention
6. 72 hours after intervention
7. 96 hours after intervention

We measure highly elevated plasma C3c levels following the intervention in all patients (FIG. 13A-G). 24 hours after the intervention the C3c levels in all patients have dropped to the baseline level before the intervention. This suggests that the patients undergo a systemic inflammatory response after the intervention and that C3c might be a highly relevant acute phase marker for systemic inflammation with a half-life that is much less than 24 hours. In addition to this, two of the patients (1026 and 1030) experience an increase in the C3c levels 72 and 96 hours after the intervention. These patients are the same patients that were later classified with SIRS (systemic inflammatory response syndrome).

### REFERENCES

1. Walport, M. J., Complement. First of two parts. N Engl J Med, 2001. 344(14): p. 1058-66.
2. Walport, M. J., Complement. Second of two parts. N Engl J Med, 2001. 344(15): p. 1140-4.
3. Walport, M. J., Complement and systemic lupus erythematosus. Arthritis Res, 2002. 4 Suppl 3: p. S279-93.
4. Jack, C., et al., Microglia and multiple sclerosis. J Neurosci Res, 2005. 81(3): p. 363-73.
5. Li, K., S. H. Sacks, and W. Zhou, The relative importance of local and systemic complement production in ischaemia, transplantation and other pathologies. Mol Immunol, 2007. 44(16): p. 3866-74.
6. Tack, B. F., et al., Evidence for presence of an internal thiolester bond in third component of human complement. Proc Natl Acad Sci USA, 1980. 77(10): p. 5764-8.
7. Seelen, M. A., et al., Functional analysis of the classical, alternative, and MBL pathways of the complement system: standardization and validation of a simple ELISA. J Immunol Methods, 2005. 296(1-2): p. 187-98.
8. Mayer, M., Complement and complement fixation. In: Kabat E A, Mayer M M, editors. Springfield, Ill.: Charles C. Thomas. Experimental immunochemistry, 1961: p. 133-240.
9. Brandslund, I., et al., Double-decker rocket immunoelectrophoresis for direct quantitation of complement C3 split products with C3d specificities in plasma. J Immunol Methods, 1981. 44(1): p. 63-71.
10. Mollnes, T. E., et al., Quantification of the terminal complement complex in human plasma by an enzyme-linked immunosorbent assay based on monoclonal antibodies against a neoantigen of the complex. Scand J Immunol, 1985. 22(2): p. 197-202.
11. Malmsten, M. and A. Schmidtchen, Antimicrobial C3a-biology, biophysics, and evolution. Adv Exp Med Biol, 2007. 598: p. 141-58.
12. Sahu, A. and J. D. Lambris, Structure and biology of complement protein C3, a connecting link between innate and acquired immunity. Immunol Rev, 2001. 180: p. 35-48.
13. Kohler, G. and C. Milstein, Continuous cultures of fused cells secreting antibody of predefined specificity. Nature, 1975. 256(5517): p. 495-7.
14. Pfeiffer, N. E., D. E. Wylie, and S. M. Schuster, Immunoaffinity chromatography utilizing monoclonal antibodies. Factors which influence antigen-binding capacity. J Immunol Methods, 1987. 97(1): p. 1-9.
15. Palarasah, Y., et al., On the effect of sodium polyanethole sulphonate (SPS) as an inhibitor of activation of complement function in blood culture systems. J Clin Microbiol, 2009.
16. Skjoedt, M. O., et al., MBL-associated serine protease-3 circulates in high serum concentrations predominantly in complex with Ficolin-3 and regulates Ficolin-3 mediated complement activation. Immunobiology, 2009.
17. Rasmussen, J. M., et al., A family with complement factor I deficiency. Scand J Immunol, 1986. 23(6): p. 711-5.
18. Pfeifer, P. H., M. S. Kawahara, and T. E. Hugli, Possible mechanism for in vitro complement activation in blood and plasma samples: futhan/EDTA controls in vitro complement activation. Clin Chem, 1999. 45(8 Pt 1): p. 1190-9.
19. Goldknopf, I. L., et al., Complement C3c and related protein biomarkers in amyotrophic lateral sclerosis and Parkinson's disease. Biochem Biophys Res Commun, 2006. 342(4): p. 1034-9.
20. Dowling, P., et al., 2-D difference gel electrophoresis of the lung squamous cell carcinoma versus normal sera demonstrates consistent alterations in the levels of ten specific proteins. Electrophoresis, 2007. 28(23): p. 4302-10.
21. Correale, M., et al., Acute phase proteins in atherosclerosis (acute coronary syndrome). Cardiovasc Hematol Agents Med Chem, 2008. 6(4): p. 272-7.
22. Mikkel-Ole Skjødt, Jette Brandt and Lars Vitved C3c as a fluid phase marker for inflammation—generation and characterization of monoclonal antibodies against neoepitopes on activated or inactivated split products of C3—Conference Information: 11th European Meeting on Complement in Human Disease, SEP 08-11, 2007 Cardiff, WALES, MOLECULAR IMMUNOLOGY Volume: 44 Issue: 16, Pages: 3917-3918.
23. Hugo F, Jenne D, Bhakdi S. Monoclonal antibodies against neoantigens of the terminal CSb-9 complex of human complement. —Biosci Rep. 1985 Aug; 5(8):649.58.
24. Scott Henwick, Seth V.Hetherington and Margaret K. Hostetter. SPECIFICITY OF 3 ANTICOMPLEMENT FACTOR-III MONOCLONAL ANTIBODIES—JOURNAL OF IMMUNOLOGICAL METHODS—Volume: 153 Issue: 1-2 Pages: 173-184 Published: AUG. 30 1992.

1. Antibody directed against an individual epitope in the C3c region of C3, characterized by immunochemically reacting with an epitope in the C3c region of native C3, said epitope having the sequence of SEQ ID NO 1.

2. Monoclonal antibody specific to the epitope of the C3c region of human C3 having the sequence of SEQ ID NO 1.

3. Use of the monoclonal antibody according to claim 1 or 2 for measurement of C3c concentration in a biological sample.

4. Use according to claim 3 for measurement in body fluids.

5. Use according to claim 4 for prognosis and diagnosis of acute and chronic inflammatory diseases including: Acute reactions such as sepsis and systemic inflammatory response syndrome (SIRS) and chronic disease state such as chronic obstructive lung disease (COLD) and immunodeficiencies (e.g factor H and factor I deficiency).

6. Use according to any of claims 3 to 5 in a multianalytic assay.

7. Use of the monoclonal antibody according to claim 2 for purification of preparations containing C3c.

8. Method for immunoassay detection and quantification of C3c comprising the steps of: 1) contacting a sample containing C3c with an antibody of claim 1 or 2 to form a complex between the antibody and C3c in an amount that is related to the amount of C3c, and 2) determining the amount of complex formed and relating the amount found to the amount of C3c in the sample.

9. A therapeutic composition comprising a therapeutically effective amount of the antibody according to claim 1 or 2 in a pharmaceutically acceptable carrier substance.

10. A method for treating of a patient with a need to decrease the amount of C3c by administering the antibody of claim 1 or 2.

11. A hybridoma cell line which produces a monoclonal antibody according to claim 2.

12. Production of an antibody possessing specificity for the epitope in the C3c region human native C3 having the sequence of SEQ ID NO 1, characterized in that cells potentially capable of producing antibodies are caused to secrete the antibody whereupon the antibody is purified and optionally isolated and/or derivatized.

13. Isolated immunogene having a sequence of SEQ ID NO 2.

14. Isolated epitope having a sequence of SEQ ID NO 1.


SEQUENCE LISTING (2 sequences)

SEQ ID NO: 1 - protein, 9 aa, Homo sapiens
  PEPTIDE (1)..(9)
CLDPERLGR

SEQ ID NO: 2 - protein, 16 aa, Homo sapiens
  PEPTIDE (1)..(16)
NKTVAVRTLDPERLGR